SEQUENCE LISTING (25 sequences)

SEQ ID NO: 1 - DNA, 45 bp, Artificial Sequence
  Description of Artificial Sequence Synthetic oligonucleotide
ggaggcgtgcggtctccgcgcgcaggttcaaatcctgcaaacccc

SEQ ID NO: 2 - DNA, 45 bp, Artificial Sequence
  Description of Artificial Sequence Synthetic oligonucleotide
ggaggcgtgcggtctccgcgcgcaggaagaaatcctgcaaacccc

SEQ ID NO: 3 - DNA, 30 bp, Artificial Sequence
  Description of Artificial Sequence Synthetic oligonucleotide
ggaggcgcaggttcaaatcctgcaaacccc

SEQ ID NO: 4 - DNA, 30 bp, Artificial Sequence
  Description of Artificial Sequence Synthetic oligonucleotide
ggaggcgcaggaagaaatcctgcaaacccc

SEQ ID NO: 5 - DNA, 32 bp, Artificial Sequence
  Description of Artificial Sequence Synthetic oligonucleotide
cctacaggcaggttcaaatcctgcagccttca

SEQ ID NO: 6 - DNA, 32 bp, Artificial Sequence
  Description of Artificial Sequence Synthetic oligonucleotide
cctacaggcaggaagaaatcctgcagccttca

SEQ ID NO: 7 - DNA, 47 bp, Artificial Sequence
  Description of Artificial Sequence Synthetic oligonucleotide
cctacaggtgcggtctccgcgcgcaggttcaaatcctgcagccttca

SEQ ID NO: 8 - DNA, 47 bp, Artificial Sequence
  Description of Artificial Sequence Synthetic oligonucleotide
cctacaggtgcggtctccgcgcgcaggaagaaatcctgcagccttca

SEQ ID NO: 9 - DNA, 34 bp, Artificial Sequence
  Description of Artificial Sequence Synthetic oligonucleotide
cattaacaggcaggttcaaatcctgctacagcca

SEQ ID NO: 10 - DNA, 34 bp, Artificial Sequence
  Description of Artificial Sequence Synthetic oligonucleotide
cattaacaggcaggaagaaatcctgctacagcca

SEQ ID NO: 11 - DNA, 49 bp, Artificial Sequence
  Description of Artificial Sequence Synthetic oligonucleotide
cattaacaggtgcggtctccgcgcgcaggttcaaatcctgctacagcca

SEQ ID NO: 12 - DNA, 49 bp, Artificial Sequence
  Description of Artificial Sequence Synthetic oligonucleotide
cattaacaggtgcggtctccgcgcgcaggaagaaatcctgctacagcca

SEQ ID NO: 13 - RNA, 33 nt, Homo sapiens
cuguuaaugcuaaucgugauagggguuuuugcc

SEQ ID NO: 14 - RNA, 32 nt, Homo sapiens
uccaacugacuccuacauauuagcauuaacag

SEQ ID NO: 15 - RNA, 32 nt, Gallus gallus
uguuaaugcuaaucgugauagggguuuuuacc

SEQ ID NO: 16 - RNA, 31 nt, Gallus gallus
ucugaaugacuccuacauguuagcauuaaca

SEQ ID NO: 17 - RNA, 34 nt, Mus musculus
cuguuaaugcuaauugugauagggguuuuggccu

SEQ ID NO: 18 - RNA, 31 nt, Mus musculus
cugacugacuccuaccuguuagcauuaacag

SEQ ID NO: 19 - RNA, 65 nt, Homo sapiens
cuguuaaugcuaaucgugauagggguuuuugccuccaacugacuccuacauauuagcauuaacag

SEQ ID NO: 20 - DNA, 300 bp, Homo sapiens
cagctttataaccgcatgtgcatacacaaacatttctttctctcttgcaggtggcacaaaccaggaaggggaaatctgtggtttaaattctttatgcctcatcctctgagtgctgaaggcttgctgtaggctgtatgctgttaatgctaatcgtgataggggtttttgcctccaactgactcctacatattagcattaacagtgtatgatgcctgttactagcattcacatggaacaaattgctgccgtgggaggatgacaaagaagcatgagtcaccctgctggataaacttagacttc

SEQ ID NO: 21 - DNA, 301 bp, Mus musculus
tttaaaaaagaaatactatcagtgctgcaaaccaggaaggggaagtgtgtggtttaagttgcatatcccttatcctctggctgctggaggcttgctggaaggctgtatgctgttaatgctaattgtgataggggttttggcctctgactgactcctacctgttagcattaacaggacacaaggcctgttactagcactcacatggaacaaatggccaccgtgggaggatgacaagtccaagagtcaccctgctggatgaacgtagatgtcagactctatcatttaatgtgctagtcataac

SEQ ID NO: 22 - DNA, 30 bp, Artificial Sequence
  Description of Artificial Sequence Synthetic oligonucleotide
gaggcgaagguuagaauccuucaaacccct

SEQ ID NO: 23 - DNA, 30 bp, Artificial Sequence
  Description of Artificial Sequence Synthetic oligonucleotide
gaggcgaagguaagauuccuucaaacccct

SEQ ID NO: 24 - DNA, 31 bp, Artificial Sequence
  Description of Artificial Sequence Synthetic oligonucleotide
taacaggaagguuagaauccuuctacagcct

SEQ ID NO: 25 - DNA, 31 bp, Artificial Sequence
  Description of Artificial Sequence Synthetic oligonucleotide
taacaggaagguaagauuccuuaagauuccu

(19) United States
(12) Patent Application Publication
(10) Pub. No.: US 2010/0292099 A1
(43) Pub. Date: Nov. 18, 2010

(54) TARGETING OF RNA WITH EXTERNAL GUIDE SEQUENCES

(75) Inventors: David H. Dreyfus, New Haven, CT (US); Lucy Y. Ghoda, San Francisco, CA (US)

Correspondence Address: WILSON, SONSINI, GOODRICH & ROSATI, 650 PAGE MILL ROAD, PALO ALTO, CA, 94304-1050, US

(73) Assignee: Keren Pharmaceutical, Inc.

(21) Appl. No.: 12/671,498

(22) PCT Filed: Aug. 22, 2008
(86) PCT No.: PCT/US08/74130

§ 371 Date: Jul. 28, 2010

Related U.S. Application Data
(60) Provisional application No. 60/957,663, filed on Aug. 23, 2007.
(60) Provisional application No. 60/957,655, filed on Aug. 23, 2007.

(51) Int. Cl. C40B 30/06; C07H 21/00; C12N 15/64; C12N 15/63; C40B 40/08; C12N 1/21; C12N 5/10

(52) U.S. Cl. 506/10; 536/23.1; 435/440; 435/320.1; 506/17; 435/252.3; 435/419; 435/348

(57) ABSTRACT

The present disclosure provides compositions and methods for modulating gene expression by using EGS to target miRNA. Another aspect of the present disclosure is the use of EGS to target mitochondrial RNA. MiRNA targets may include immature or mature forms of miRNA, such as miRNA overexpressed in diseases such as cancer.

### CROSS-REFERENCE TO RELATED APPLICATIONS

This application claims the priority benefit of U.S. Provisional Application Ser. Nos. 60/957,655 filed on Aug. 23, 2007, and 60/957,663, filed Aug. 23, 2007, pending, which are hereby incorporated herein by reference in their entirety.

### BACKGROUND

RNaseP is found in all organisms and is responsible for the maturation of 5′ termini of all tRNAs. RNaseP recognizes its substrate by structure rather than by sequence. As a result, any two RNA molecules that form a structure that mimics a precursor tRNA will elicit cleavage by RNaseP. An external guide sequence (EGS) may be designed to mimic this structure by forming a complex with a target RNA. The targeted structure and EGS would thus form a substrate that is cleaved by RNaseP. A suitably designed EGS can be targeted to cleave an mRNA species, such as that of the IL-4Rα and the influenza virus (Dreyfus et al., International Immunopharmacology, 4:1015-1027 (2004); Plehn-Dujowich and Altman, PNAS 95:7327-7332 (1998)). Nuclease resistant EGS has also been designed and introduced exogenously to cleave intracellular targets (Ma et al., Nature Biotech. 18:58-61 (2000)).

EGS may also be designed to target other RNA species such as microRNA (miRNA). MicroRNA is a recently discovered population of non-coding small RNA molecules belonging to a class of regulatory molecules found in plants and animals that control gene expression by binding to complementary sites on target messenger RNA (mRNA) transcripts. MiRNA are about 15-50 nucleotides in length and has a role in regulating gene expression in eukaryotic organisms.

Endogenous miRNAs are embedded in independent non-coding RNAs or in introns of protein-coding genes and are transcribed as long primary transcripts (pri-miRNA) by RNA polymerase II and typically contains one or more local double-stranded or “hairpin” regions and the 5′ cap and polyadenylated tail of an mRNA. These large RNA precursors, pri-miRNAs, can be several kilobases in length, with one or more hairpin regions. The pri-miRNAs are then processed in the nucleus into approximately 60-70 nucleotide pre-miRNAs, by Drosha, which fold into imperfect stem-loop structures (Lee et al., Nature 425:415-9 (2003)). Recently, an alternative pathway has been reported in which debranched introns mimic the structure of pre-miRNA. Thus, it is believed this pathway does not require Drosha processing to enter the next stage of generating mature miRNA, in which the pre-miRNAs undergo a processing step within the cytoplasm. The processing of pre-miRNA in the cytoplasm generates mature miRNAs of 18-25 nucleotides in length, by excision from one side of the pre-miRNA hairpin by an RNase III enzyme, Dicer (Hutvagner et al., Science 293:834-8 (2001) and Grishok et al., Cell 106:23-34 (2001)).

It has been demonstrated that miRNAs regulate gene expression in two ways. The first is by miRNAs binding the protein-coding mRNA sequences that are complementary to the miRNA induce the RNA-mediated interference (RNAi) pathway. The mRNA targets are cleaved by ribonucleases in the RISC complex. This mechanism of miRNA-mediated gene silencing has been observed mainly in plants (Hamilton and Baulcombe, Science 286:950-2 (1999) and Reinhart et al., Genes and Dev. 16:1616-26 (2002)), however, an example is known from animals (Yekta et al., Science 304:594-6 (2004)). This mechanistic step is analogous to siRNA, which also uses the RNAi pathway. In the second mechanism, miRNA binds to imperfect complementary sites on mRNA transcripts, typically the 3′UTR of the gene. The target is not cleaved, but translation of the gene is inhibited. This mechanism has been identified in both plants and animals (Bartel, Cell 116:281-97 (2004)).

MiRNAs are expressed in a wide variety of organisms, including worms, insects, plants and animals. It is believed the number of miRNA genes vary from 800 to over 2000 with many being conserved across mammalian species. For example, the Sanger database, http://microrna.sanger.ac.uk/, a database of miRNA data, has approximately 400 conserved regulatory miRNAs for vertebrates.

MiRNAs appear to be a major feature of the gene regulatory network. MiRNAs have been implicated in regulating cellular differentiation, proliferation and apoptosis, as well as in development, metabolism, embryogenesis and patterning, differentiation and organogenesis. As a result, miRNAs are believed to be involved in a number of human diseases, for example in cancer and viral infections. For example, it has been shown that the miRNA profiles are altered in a number of cancers, where miRNAs may function variously as oncogenes or as tumor suppressors (O'Donnell et al., Nature, 435:839-843 (2005); Cai et al., Proc. Natl. Acad. Sci. USA, 102:5570-5575 (2005); Morris and McManus, Sci. STKE, pe41 (2005)).

MiRNA has been proposed to modulate gene expression, and may also be expressed in a tissue-specific and developmental stage-specific manner. MiRNA gene families are estimated to account for at least 1% of some genomes, modulating the expression of approximately a third of all genes (Tomari et al., Curr. Biol., 15:R61-64 (2005); Tang, Trends Biochem. Sci., 30:106-14 (2005); Kim, Nature Rev. Mol. Cell. Biol., 6:376-385 (2005)).

Because miRNAs are important regulatory elements in eukaryotes, controlling miRNA expression provides a means of modulating gene expression.

### SUMMARY

The present disclosure provides compositions and methods for modulating gene expression by using EGS to target miRNA. Another aspect of the present disclosure is the use of EGS to target mitochondrial RNA. Mitochondrial transcripts can be readily targeted by EGS as RNase P is expressed in the mitochondria.

One aspect of the present disclosure is an external guide sequence (EGS) comprising an oligonucleotide designed to target a miRNA. The miRNA may be an immature or mature form of miRNA, for example, the immature miRNA may be a pri-miRNA or pre-miRNA. The EGS may comprise a subcellular localization sequence. In some embodiments, the subcellular localization sequence is a nuclear localization element. In one aspect of this embodiment, the nuclear localization element is a hexamer sequence. In another distinct but related aspect, the subcellular localization sequence is a mitochondrial localization sequence. In some embodiments, the mitochondrial localization sequence comprises a peptide or an oligonucleotide.

The miRNA may regulate apoptosis, fat metabolism, development, differentiation, proliferation, or stress response. In other embodiments, the miRNA is overexpressed in a disease selected from the group of immune disease, neurological disease, developmental disease, cardiovascular, skeletal disease, or cancer. The cancer may be selected from the group consisting of: leukemia, lymphoma, gastric cancer, lung cancer, and prostate cancer. The miRNA may be encoded from the miR-17-92 cluster or miR-106-363 cluster. The miRNA may be miR-21, miR-150, miR-155, miR-375, miR-1-1, miR-1-2, or miR-133. The miRNA may be overexpressed and secreted by tumor cells.

In another embodiment, the miRNA is a viral miRNA. In some embodiments, the viral miRNA is from rotavirus, influenza virus, parainfluenza virus, respiratory synctyial virus, herpes virus, Flavivirus, human immunodeficiency virus, hepatitis virus, human papillomavirus, Epstein-Barr virus, Ebola virus, Rous sarcoma virus, human rhinovirus, Variola virus, and poliovirus. The influenza virus may be influenza A, influenza B, or influenza C. The influenza virus may be of the H1N1, H2N2, H3N2, H5N1, H7N7, H1N2, H9N2, H7N2, H7N3, or H10N7 serotype. In other embodiments, the viral miRNA is Kaposi's sarcoma herpesvirus.

The present disclosure further provides a method of modulating gene expression in a host cell comprising contacting the host cell with an EGS comprising an oligonucleotide designed to target a miRNA, wherein the miRNA may be an immature or mature form of miRNA. For example, the immature miRNA may be a pri-miRNA or pre-miRNA. Contact with the EGS can cause a change in expression of a gene in the host cell in comparison to expression of the gene in a host cell not in contact with the EGS.

In yet another aspect of the present disclosure, an external guide sequence comprising an oligonucleotide designed to target a mitochondrial RNA is provided. In some embodiments, the oligonucleotide comprises a mitochondrial localization sequence. In one aspect of the embodiment, the mitochondrial localization sequence comprises a peptide or an oligonucleotide. In some embodiments, the mitochondrial RNA is derived from a mitochondrial gene causing a dysfunctional electron transport chain. In other embodiments, the mitochondrial RNA is derived from a mitochondrial gene causing a disease of the brain, muscle, nerve, heart, pancreas, eye, ears, kidney, or gastrointestinal system.

The present disclosure also provides a method of modulating gene expression in a host cell comprising contacting the host cell with an EGS comprising an oligonucleotide designed to target a mitochondrial RNA, wherein contact with the EGS causes a change in expression of a gene in the host cell in comparison to expression of the gene in a host cell not in contact with the EGS.

Further provided in the present disclosure are vectors and host cells comprising the external guide sequences described herein. In some embodiments, host cells comprise the vectors of the present disclosure. The vectors of the present disclosure may comprise a regulatory element.

In yet another aspect of the present disclosure, a library of EGS comprising a plurality of oligonucleotides designed to target a plurality of miRNA is provided. The plurality of miRNA, or a subset thereof, may be an immature or mature form of miRNA, for example, the immature miRNA may be a pri-miRNA or pre-miRNA. The plurality of oligonucleotides may comprise a plurality of subcellular localization sequences. The subcellular localization sequences may be nuclear localization elements. In one preferred embodiment, the nuclear localization element is a hexamer sequence. In other embodiments, the subcellular localization sequences are mitochondrial localization sequences. The subcellular localization sequences may be peptides or oligonucleotides.

Also provided in the present disclosure is a method of identifying an EGS that modulates expression of a gene comprising: (a) contacting a host cell with a library of EGS comprising a plurality of oligonucleotides designed to target a plurality of miRNA in the host cell; (b) analyzing a gene expression profile of the host cell to determine the gene whose expression is modulated by contact with the library of EGS; and (c) identifying the EGS within the library that modulates the expression of the gene. In another aspect, the method comprises identifying the gene being modulated by the EGS library.

In another aspect of the present disclosure, a library of EGS comprising a plurality of oligonucleotides designed to target a plurality of mitochondrial RNA is provided. The plurality of oligonucleotides may comprise a plurality of subcellular localization sequences. In some embodiments, the subcellular localization sequences are mitochondrial localization sequences. In other embodiments, the subcellular localization sequences are peptides or oligonucleotides.

In yet another aspect provided by the present disclosure is a method of identifying an EGS that modulates expression of a gene comprising: (a) contacting a host cell with a library of EGS comprising a plurality of oligonucleotides designed to target a plurality of mitochondrial RNA in the host cell; (b) analyzing a gene expression profile of the host cell to determine the gene whose expression is modulated by contact with the library of EGS; and (c) identifying the EGS within the library that modulates the expression of the gene.

In some embodiments of the methods provided herein, the methods may further comprise providing analysis or identification of the EGS to an individual. The methods may comprise providing analysis or identification of the EGS to an individual comprising transmission of the data relating to the analysis or identification over a network.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a schematic representation of an EGS targeting miRNA and its effect on the miRNA gene target.

FIG. 2 is a schematic representation of an EGS library promoting apoptosis.

FIG. 3 is a block diagram of a representative system for analyzing data and providing identification of an EGS from cell-based assays over a network.

FIG. 4 depicts predicted or confirmed miR-155 transcript and stem-loop structure in various organisms.

FIG. 5 depicts miR-155 transcript in A) human and B) mouse. A) Human pre-miR-155 sequence is depicted along with the mature miR-155 sequence. The miR-155 transcript (BIC) is also shown with a number of GNNNNNNU, potential RNaseP sites, underlined (and further listed in a box on the left), numbered 2.1, 2.2, 3.1, 3.2, 1.3, 1.4, 4.1, and 4.2. The pre-form is in parenthesis and in bold italics. The mature miR-155 is in brackets. B) Mouse miR-155 transcript is depicted and compared to human miR-155 transcript. Potential RNaseP sites are underlined and number 3.1 and 3.2, and the expected pre-form of miR-155 is in brackets, and is in bold italics.

FIG. 6 depicts RNA based EGS sequences for miR-155. A) RNA based EGS sequence based on potential RNaseP site 1.3 (see FIG. 5A) with FITC attached at the 5′ end and B) the corresponding T-loop mutant as a negative control. C) RNA based EGS sequence based on potential RNaseP site 3.2 (see FIG. 5A) (WT miR 155 3.2) and the corresponding T-loop mutant (Mt miR-155 3.2) as a negative control

FIG. 7 is a bar graph illustrating the effect of EGS inhibition on miR-155.

### DETAILED DESCRIPTION

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

General Techniques:

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Definitions:

As used in the specification and claims, the singular form “a”, “an” and “the” include plural references unless the context clearly dictates otherwise. For example, the term “a cell” includes a plurality of cells, including mixtures thereof.

The terms “polypeptide”, “peptide”, “amino acid sequence” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this disclosure.

“Linked” refers to the joining together of two more chemical elements or components, by whatever means including chemical conjugation or recombinant means. “Linked” also refers to a juxtaposition wherein the components so described are in a relationship permitting them to function in their intended manner. For instance, a promoter sequence is linked to a coding sequence if the promoter sequence promotes transcription of the coding sequence.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide which is known to comprise additional residues in one or both directions.

“Heterologous” means derived from a genotypically distinct entity from the rest of the entity to which it is being compared. For example, a promoter removed from its native coding sequence and linked to a coding sequence other than the native sequence is a heterologous promoter. The term “heterologous” as applied to a polynucleotide, a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

A “subcellular localization sequence” as applied to polynucleotide or polypeptide of the subject disclosure refers to a sequence that facilitates transporting or confining a protein to a defined subcellular location. Defined subcellular locations include extracellular space (occupied by e.g. secreted proteins), nucleus, endoplasmic reticulum (ER), Golgi apparatus, coated pits, mitochondria, endosomes, and lysosomes.

The terms “cytosolic”, “nuclear” and “mitochondrial” as applied to cellular proteins specify the extracellular and/or subcellular location in which the cellular protein is mostly, predominantly, or preferentially localized.

As used herein, “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectively referred to as gene product. If the polynucleotide is derived from genomic DNA, expression may include splicing of the mRNA in a eukaryotic cell.

A “cell line” or “cell culture” denotes bacterial, plant, insect or higher eukaryotic cells grown or maintained in vitro. The descendants of a cell may not be completely identical (either morphologically, genotypically, or phenotypically) to the parent cell.

The terms “gene” or “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

A “vector” is a nucleic acid molecule, preferably self-replicating, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions.

An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

External Guide Sequence (EGS)

One aspect of the present disclosure is the design of an external guide sequence (EGS). The subject EGS is particularly suitable for targeting an RNA molecule. In preferred embodiments, the RNA moleule is miRNA, such as mature or immature miRNA, the latter which may include pre-miRNA or pri-miRNA. In other embodiments, the RNA molecule is mitochondrial RNA, in particular mitochondrial mRNA. In another embodiment, the EGS may target the promoter of the miRNA or mitochondrial mRNA. The subject EGS exhibits one or more unique features that are described below.

An EGS is typically designed to base pair through hydrogen bonding mechanism with a target RNA molecule to form a molecular structure similar to that of a transfer RNA (tRNA). The EGS/RNA target is then cleaved and inactivated by RNase P. EGS is generally designed to mimic certain structural features of a tRNA molecule when it forms a bimolecular complex with another RNA sequence. Thus, any RNA sequence can in principle be recognized as a substrate for RNase P with both the EGS and RNase P participating as cocatalysts. Design of an EGS typically involves both knowledge of the RNA primary sequence to be cleaved by RNase P as well as the secondary structure, if any, of the RNA sequence. Secondary structure can be approximated by computer modeling programs (Zuker, Nucl. Acids Res. 31:3406-3415 (2003)) or databases available online with predicted structures, such as the Sanger database for miRNA (http://microrna.sanger.ac.uk/sequences/). Secondary structure can also be determined empirically using nucleases or other RNA cleaving reagents well known to one of ordinary skill in the art.

EGS sequences generally exhibit sequence complementarity to the primary sequence of the targeted RNA. For example, the portion of EGS designed to hybridize with the primary sequence of the targeted RNA may be at least 50%, 60%, 70%, 80%, 90%, or 100% complementary to the targeted RNA sequence. The sequences in the RNA is typically exposed in a single-stranded conformation within the RNA secondary structure in order to bind to the EGS. EGS for promoting RNAase P-mediated cleavage of RNA has been developed for use in eukaryotic systems as described by U.S. Pat. No. 5,624,824 to Yuan, et al., U.S. Pat. No. 6,610,478 to Takle, et al., WO 93/22434 to Yale University, WO 95/24489 to Yale University, and WO 96/21731 to Innovir Laboratories, Inc. In eukaryotes, including mammals, tRNAs are usually encoded by families of genes that are usually about 70 to 200 base pairs long, preferably about 70 to 150 base pairs long. tRNAs generally assume a secondary structure with four base paired stems known as the cloverleaf structure. The tRNA typically contains a stem, a D loop, a Variable loop, a TΨX loop, and an anticodon loop. In one form, the EGS contains at least seven nucleotides which base pair with the target sequence 3′ to the intended cleavage site to form a structure like the stem, nucleotides which base pair to form stem and loop structures similar to the TΨC loop, the Variable loop and the anticodon loop, followed by at least three nucleotides that base pair with the target sequence to form a structure like the D loop.

Preferred EGS for eukaryotic RNAase P comprises a sequence which, when in a complex with the target RNA molecule, forms a secondary structure resembling that of a tRNA cloverleaf or parts thereof. The desired secondary structure may be determined using conventional Watson-Crick base pairing schemes to form a structure resembling a tRNA. Since RNase P generally recognizes structures as opposed to sequences, the specific sequence of the hydrogen bonded regions is less critical than the desired structure to be formed. The EGS and the target RNA substrate typically resembles a sufficient portion of the tRNA secondary and tertiary structure to result in cleavage of the target RNA by RNAase P. The sequence of the EGS can be derived from any tRNA. The sequences and structures of a large number of tRNAs are well known to one of ordinary skill in the art and can be found at least at: http://rna.wustl.edu/tRNAdb-/.

A consensus sequence for RNase P recognition of tRNA molecules is GNNNNNU, however, The EGS of the present disclosure may also target sequences without the GNNNNNU consensus sequence. The sequence obtained from the stem of the tRNA may be altered to be complementary to the identified target RNA sequence. Target RNA may be mapped by techniques well known to one of ordinary skill in the art for the consensus sequence. Such techniques include digestion of the target RNA with T1 nuclease. For example, digestion with T1 nuclease cleaves RNA after guanine (G) residues that are exposed in solution and single-stranded, but not after G residues that are buried in the RNA secondary structure or base paired into double-stranded regions. The reaction products may form a ladder after size fractionation by gel-electrophoresis. A T1 sensitive site may be detected as a dark band is compared to the target RNA sequence to identify RNase P consensus sequences. The complementary sequence from the target RNA may then be used for the EGS. The complementary sequences may consist of at least 4, 5, or 6 nucleotides. In preferred embodiments, it may consist of as few as seven nucleotides, but preferably include eleven nucleotides, in two sections which base pair with the target sequence and which are preferably separated by two unpaired nucleotides in the target sequence, preferably UU, wherein the two sections are complementary to a sequence 3′ to the site targeted for cleavage. The remaining portion of the guide sequence, which is believed to cause RNAase P catalytic RNA to interact with the EGS/target RNA complex, is herein referred to as an RNAase P binding sequence. The anticodon loop and the variable loop can be deleted and the sequence of the TΨC loop can be changed without decreasing the usefulness of the guide sequence. External guide sequences can also be derived using in vitro evolution techniques (see U.S. Pat. No. 5,624,824 to Yuan, et al. and WO 95/24489 to Yale University).

The use of EGS to target RNA molecules and thereby modulate the expression of cellular targets has advantages over both the use of RNAi or antisense DNA oligonucleotides. RNase P is present in abundant quantities in all viable eukaryotic cells where it serves to process tRNA by cleavage of a precursor transcript. EGS are generally not consumed in this reaction, but instead can recycle as a catalyst through multiple cycles of target RNA cleavage leading to target inactivation more effectively than conventional antisense DNA oligonucleotides. EGS can combine the specificity of conventional antisense DNA for gene targeting with the catalytic potency of RNase P. As RNase P is required for all replication in cells, it is ubiquitous, unlike RISC targeting in RNAi. EGS will generally also have less non-specific effects and less non-specific inflammatory effects than comparable gene targeting with RNAi or conventional antisense DNA. Without being bound by any particular theory, less non-specific effects and less non-specific inflammatory effects of EGS may in part be due to 1) EGS typically being significantly smaller than comparable RNAi, 2) EGS generally having significantly less double stranded RNA than RNAi, the latter being capable of triggering Toll-3 innate immune receptors, 3) EGS generally not having DNA CpG motifs present in DNA based antisense, the latter being capable of triggering Toll-9 innate immune receptors, and 4) EGS are based on activation of RNase P, a housekeeping enzyme not induced or regulating the host anti-viral response in contrast to RNAi activated RISC and double strand DNA activated RNase H. Previous studies have also demonstrated that EGS-based RNA inactivation of targeted mRNA in vivo can be orders of magnitude more effective than gene inactivation by antisense DNA oligonucleotides (Guerrier-Takada and Altman, Methods Enzymol. 313:442-456 (2000); Plehn-Dujowich and Altman, PNAS 95:7327-7332 (1998)).

Other aspects of the present disclosure include the design of EGS that are nuclease resistant. Chemical modifications may be made which greatly enhance the nuclease resistance of the heterologous sequence without compromising their biological function of inducing or catalyzing cleavage of the RNA target. Chemical modifications include modification of the phosphodiester bonds of the heterologous sequence, e.g. to methylphosphonate, the phosphotriester, the phosphorothioate, the phosphorodithioate, or the phosphoramidate, so as to render the heterologous sequence more stable in vivo. The naturally occurring phosphodiester linkages in oligonucleotides may be susceptible to degradation by endogenously occurring cellular nucleases, while many analogous linkages are highly resistant to nuclease degradation. The use of a “3′-end cap” strategy by which nuclease-resistant linkages are substituted for phosphodiester linkages at the 3′ end of the oligonucleotide may protect oligonucleotides from degradation (Tidd and Warenius, Br. J. Cancer 60:343-350 (1989); Shaw et al., Nuclx Acids Res. 19:747-750 (1991)). Phosphoroamidate, phosphorothioate, and methylphosphonate linkages all function adequately in this manner. More extensive modification of the phosphodiester backbone has been shown to impart stability and may allow for enhanced affinity and increased cellular permeation of oligonucleotides. Many different chemical strategies have been employed to replace the entire phosphodiester backbone with novel linkages. The analogues of the oligonucleotides of the disclosure include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene(methylimino) (MMI) or methyleneoxy(methylimino) (MOMI) linkages. Phosphorothioate and methylphosphonate-modified oligonucleotides are particularly preferred because of their availability for automated oligonucleotide synthesis. For example, one or more of the bases of an EGS can be replaced by 2′ methoxy ribonucleotides or phosphorothioate deoxyribonucleotides using available nucleic acid synthesis methods well known to one of ordinary skill in the art. Synthesis methods are described by, for example, PCT WO 93/01286 by Rosenberg et al. (synthesis of sulfurthioate oligonucleotides); Agrawal et al., Proc. Natl. Acad. Sci. USA 85:7079-7083 (1988); Sarin et al., Proc. Natl. Acad. Sci. USA 85:7448-7794 (1989); Shaw et al., Nucl. Acids Res. 19:747-750 (1991) (synthesis of 3′ exonuclease-resistant oligonucleotides containing 3′ terminal phosphoroamidate modifications).

Degradation of oligonucleotide analogues is mainly attributable to 3′-exonucleases. Various 3′-modifications known in the art can greatly decrease the nuclease susceptibility of these analogues such as introduction of a free amine to a 3′ terminal hydroxyl group of the oligonucleotide. Cytosines in the sequence can be methylated, or an intercalating agent, such as an acridine derivative, can be covalently attached to a 5′ terminal phosphate to reduce the susceptibility of a nucleic acid molecule to intracellular nucleases. Chemical modifications also include modification of the 2′ OH group of a nucleotide's ribose moiety, which has been shown to be critical for the activity of the various intracellular and extracellular nucleases. Typical 2′ modifications include, but are not limited to, the synthesis of 2′-O-Methyl oligonucleotides, as described by Paolella et al., EMBO J. 11: 1913-1919 (1992), and 2′-fluoro and 2′-amino-oligonucleotides, as described by Pieken et al., Science 253: 314-317 (1991), and Heidenreich and Eckstain, J. Biol. Chem. 267: 1904-1909 (1992). Portions of the EGS can also contain deoxyribonucleotides, which improve nuclease resistance by eliminating the critical 2′ OH group. Nuclease resistant EGS as described above can also be obtained from suppliers such as Dharmacon (Boulder, Colo.). The EGS can be RNA or DNA, or modified derivatives thereof. The EGS can be produced artificially, such as by chemical synthesis, or by a living organism, such as in bacteria. Methods of producing the EGS include in vitro transcription, PCR, vector expression, and viral expression.

The present disclosure also provides vectors of the EGS designed to target miRNA and mitochondrial mRNA. Preferred vectors for introducing EGS into mammalian cells include viral vectors, such as the retroviruses, which introduce the vector directly into the nucleus where the DNA is then transcribed to produce the encoded EGS. Examples of methods for using retroviral vectors for gene therapy are described in U.S. Pat. Nos. 4,868,116 to and 4,980,286 to Morgan et al.; PCT applications WO 90/02806 and WO 89/07136; and Mulligan, Science 260:926-932 (1993); the teachings of which are incorporated herein by reference.

Defective retroviral vectors, which incorporate their own RNA sequence in the form of DNA into the host chromosome, can be engineered to incorporate EGS into the cells of a host, where copies of the EGS will be made and released into the cytoplasm or are retained in the nucleus to interact with the target nucleotide sequences of the RNA. For example, bone marrow stem cells and hematopoietic cells can be transfected in vitro or in vivo with retrovirus-based vectors encoding EGS. Such cells are relatively easily removed and replaced from humans, and provide a self-regenerating population of cells for the propagation of transferred genes. When in vitro transfection of stem cells is performed, once the transfected cells begin producing the particular EGS, the cells can be added back to the patient to establish entire clonal populations of cells that are expressing EGS.

As an example, a vector used to clone and express DNA sequences encoding EGS may include one or more of the following:

1. A cloning site in which to insert a DNA sequence encoding an EGS molecule to be expressed.
2. A mammalian origin of replication (optional) which allows episomal (non-integrative) replication, such as the origin of replication derived from the Epstein-Barr virus.
3. An origin of replication functional in bacterial cells for producing quantities of the DNA encoding the EGS constructs, such as the origin of replication derived from the pBR322 plasmid.
4. A promoter, such as one derived from Rous sarcoma virus (RSV), cytomegalovirus (CMV), or the promoter of the mammalian U6 gene (an RNA polymerase III promoter) which directs transcription in mammalian cells of the inserted DNA sequence encoding the EGS construct to be expressed.
5. A mammalian selection marker (optional), such as neomycin or hygromycin resistance, which permits selection of mammalian cells that are transfected with the construct.
6. A bacterial antibiotic resistance marker, such as neomycin or ampicillin resistance, which permits the selection of bacterial cells that are transformed with the plasmid vector.

An exemplary vector for delivering and expressing EGS in vivo uses an RNA polymerase III (pol III) promoter for expression. Such promoters can produce transcripts constitutively without cell type specific expression. Pol III promoters also generate transcripts that can be engineered to remain in the nucleus of the cell, the location of many target RNA molecules. In one aspect, a complete pol III transcription unit may be used, including a pol III promoter, capping signal, and termination sequence. Pol III promoters and other pol III transcription signals are present in tRNA genes, 5S RNA genes, small nuclear RNA genes, and small cytoplasmic RNA genes. Preferred pol III promoters for use in EGS expression vectors are the human small nuclear U6 gene promoter and tRNA gene promoters. The use of U6 gene transcription signals to produce short RNA molecules in vivo is described by Noonberg et al., Nucleic Acids Res. 22:2830-2836 (1995), and the use of tRNA transcription signals is described by Thompson et al., Nucleic Acids Res., 23:2259-2268 (1995), both hereby incorporated by reference. The pol III promoters may be inducible, for example, based on the regulatory elements described below.

Many pol III promoters are internal, that is, they are within the transcription unit. Thus, these pol III transcripts include promoter sequences. Where desired, the promoter sequences typically do not interfere with the structure or function of the EGS. Since EGS molecules are derived from tRNA molecules, tRNA gene promoter sequences can be easily incorporated into EGS molecules. The internal promoter of tRNA genes typically occurs in two parts, an A box and a B box. In tRNA molecules, A box sequences are generally present in the D loop and half of the D stem of tRNA molecules, and B box sequences are generally present in the T loop and the proximal nucleotides in the T stem. Minimal EGS molecules typically retain the T stem and loop structure, and the B box sequences can be incorporated into this part of the EGS in the same way they are incorporated into the T stem and loop of tRNA molecules. Since a minimal EGS generally does not require a D loop or stem, A box sequences need not be present in any of the functional structures of the EGS molecule. For example, A box sequences can be appended to the 5′ end of the EGS, after the D recognition arm, such that the proper spacing between the A box and B box is maintained. The U6 gene promoter is not internal (Kunkel and Pederson, Nucleic Acids Res. 18:7371-7379 (1989); Kunkel et al., Proc. Natl. Acad. Sci. USA 83:8575-8579 (1987); Reddy et al., J. Biol. Chem. 262:75-81 (1987)). Suitable pol III promoter systems useful for expression of EGS molecules are described by Hall et al., Cell 29:3-5 (1982), Nielsen et al., Nucleic Acids Res. 21:3631-3636 (1993), Fowlkes and Shenk, Cell 22:405-413 (1980), Gupta and Reddy, Nucleic Acids Res. 19:2073-2075 (1990), Kickoefer et al., J. Biol. Chem. 268:7868-7873 (1993), and Romero and Balckburn, Cell 67:343-353 (1991). The use of pol III promoters for expression of ribozymes is also described in WO 95/23225 by Ribozyme Pharmaceuticals, Inc.

In another novel aspect of the present disclosure, the EGS may comprise a regulatory element. The regulatory element allows regulation of the modification of gene response by the EGS. For example, the EGS may not be expressed until the regulatory element is induced by a regulatory factor. The regulatory element can be present in the EGS as a separate entity or linked to the EGS. The regulatory element may be induced by regulatory factors such as light, temperature, oxygen levels, ion concentration, or injury, such as a pathological response or a wound. The regulatory factor can also be a ligand. Ligands can be synthetic or natural, such as oligonucleotides, polypeptides, proteins, polysaccharides, sugars, organic molecules and inorganic molecules. The regulatory factor can also be selected from the group of hormones, antibiotics, metals, ions, and steroids. Regulatory factors include, but are not limited to, cytokines, growth factors, and steroids. Regulatory factors also include cAMP, tetracycline, doxycycline, arabinose, ecdysone, and steroids. The regulatory element can also be in the form of a Cre-Lox system, a regulatable ribozyme, or promoter. The regulatory elements can be tissue or cell type specific and the EGS may have one or more regulatory elements.

Regulatory elements taking the form of a promoter can be chemically regulated or physically regulated. Chemically regulated promoters include promoters regulated by alcohol, tetracycline, steroids, metals, and carbohydrates. Physically regulated promoters include those regulated by temperature or light. One of ordinary skill in the art can modify existing promoter systems from bacteria, fungi, plants, and animals for use in a particular system. Promoter systems from one organism can be adapted and transferred to another. For example, the tetracyclin-regulated system from bacteria has been widely used in mammalian cells. Promoters from murine cells can be transferred to human cells. Many commercially available promoter systems are also available and can be adapted to the present disclosure. In preferred embodiments the regulatory element controls transcription of the EGS, and the regulatory element is typically a promoter. The promoter may be inducible and preferably promotes transcription of small RNA.

The regulatory elements can be induced by one or more regulatory factors and the regulatory factor can promote the production of one or more products. For example, the first regulatory element may be activated by a regulatory factor, and induces the production of EGS. The first regulatory element may also produce another product. The product and/or the EGS can provide a positive feedback loop by activating the same first regulatory element. The EGS can also comprise one or more regulatory elements. For example, the EGS can comprise both a regulatable ribozyme and a promoter, or two different promoters. In an extension of the above example, the product of a first regulatory element can activate a second regulatory element, which induces production of the same or different EGS, targeting the same or different target, effectively modulating the expression of a gene. The regulatory element can also provide negative feedback. Instead of activating the regulatory element, the product of a regulatory element can inhibit its activity. One can envision a system with more than one regulatory element in a negative feedback loop, wherein the product of the first regulatory element activates a second regulatory element, and the product from the activation of the second regulatory element inhibits the first regulatory element. Another example is the first regulatory element can drive both the production of the EGS and a second product that inhibits the first regulatory element, or that activates a second regulatory element that produces a product that inhibits the first regulatory element. Numerous combinations can be envisioned by one of ordinary skill in the art.

The regulatory element can be an alcohol regulated promoter system. A system has been adapted from the fungus Aspergillus nidulans and applied to plants (EP637339 to Syngenta Ltd.). A first promoter is linked to the AlcR encoding gene and a second promoter is linked to the target. The second promoter is one derived from the aldhehyde dehydrogenase gene or other alcohol dehydrogenase genes involved in the ethanol utilization pathway. The second promoter is activated by AlcR binding, and AlcR can bind only in the presence of alcohol, such as ethanol, ethyl methyl ketone or other alcohols/ketones. Such a system in our present disclosure may be modified such that the target is a heterologous sequence and an effective amount of alcohol, and type of alcohol, is safe to the host cell or animal being administered the EGS.

The EGS can also be regulated by temperature, through the use of an inducible heat shock promoter. External stress such as increased temperature induces heat shock factors (HSF) to interact with heat shock response elements (HSE). The interaction stimulates expression of heat shock proteins. The system can be modified for use to induce expression of other genes and used in different organisms ranging from bacteria to plants to animals (U.S. Pat. No. 7,056,897 to Tsang et al., U.S. Pat. No. 7,183,262 to Li et al.). U.S. Pat. Nos. 5,614,381, 5,646,010 and WO 89/00603 drive expression using heat shock at temperatures greater than 42° C. These temperatures are generally not practicable in human therapy as they can not be maintained for a sustained period of time without harm to the individual. Regulatory elements that may be used at temperatures of 42° C. and below, systemically or locally to treat a subject such that the expression of the EGS is activated preferentially in regions of the body that have been subjected to conditions which induce such expression. Examples of heat shock promoters include, HSP70 or HSP70B; and the heat applied to the cell may be from about basal temperature to about 42° C. As used herein, the basal temperature of the cell is defined as the temperature at which the cell is normally found in its natural state, for example, a cell in skin of a mammal may be at temperatures as low as 33° C. whereas a cell in the liver of an organism may be as high as 39° C. In specific embodiments, the application of hyperthermia involves raising the temperature of the cell from basal temperature, most typically 37° C. to about 42° C. or less. Alternatively, the hyperthermic conditions may range from about 38° C. to about 41° C. or from about 39° C. to about 40° C. Other heat shock promoters include, for example, HSP90, HSP60, HSP27, HSP72, HSP73, HSP25 and HSP28. A minimal heat shock promoter derived from HSP70 and comprising the first approximately 400 bp of the HSP70B promoter may also be used in the disclosure. In an alternative embodiment, the regulatory element comprises a hypoxia-responsive element (HRE). This hypoxia-responsive element may optionally contain at least one binding site for hypoxia-inducible factor-1 (HIF-1). The expression of the EGS may be placed under the control of the heat shock promoter and its expression may be induced when the temperature of a subject increases or hypoxic cellular conditions arise, inducing expression of the EGS.

The tetracycline-regulated promoter system can also be used as a regulatory element and is well known in the art (Gossen M, Bujard, PNAS 89:5547-5551 (1992); U.S. Pat. No. 5,851,796 to Schatz, U.S. Pat. No. 6,136,954 to Bujard). The system was derived from E. coli. In E. coli, the tetracyclin resistance operon is bound by the Tet respressor (TetR), thereby inhibiting transcription. Tetracycline binds TetR, changing its conformation, and thereby allowing transcription. Systems have been developed wherein the addition of tetracycline, or its derivative, can either activate or inhibit transcription. It has been used successfully used in plants and animals. The system has been modified such that the TetR has been mutated to be an activator of gene expression. TetR is fused to the strong activation sequence of herpes simplex virus protein 16 (VP 16), and the resulting fusion protein, tetracycline transactivator (tTA) binds the operon activating trascription. Tetracyclin binds tTA, releases the operator and therefore turning off gene trascription. For the opposite effect, tTA has a four amino acid change and is denoted rtTA. This fusion protein can recognize the operon sequence only in the presence of doxycyline, as a result, only in the presence of doxycycline there is transcription. The Tet system is easily modified for use as the regulatory element in the EGS. The EGS may be inactivated by addition of tetracycline, such that expression of the EGS is inhibited. The Tet system can act in the opposite manner, wherein the addition of tetracyclin induces the expression of the EGS.

Metal-regulated promoters can also be used as the regulatory element in the EGS. Metallothioneins are proteins that bind and sequester ionic forms of certain metals in fungi, plants, and animals. Metals include copper, zinc, cadmium, mercury, gold, silver, cobalt, nickel, and bismuth. Typically, proteins that can bind the metals contain cysteine motifs. Examples of metallothionenin promoters are known in the art, wherein the activity of the promoter is dependent on the metal ion concentration (U.S. Pat. No. 4,579,821 to Palmiter et al.; U.S. Pat. No. 4,601,978 to Karin). The expression of the EGS may be under the control of the metal-regulated promoter and with changes in the metal concentration, EGS expression may be modulated.

In other embodiments, the EGS comprises regulatory elements induced by carbohydrates, such as in the arabinose-regulated promoter system. This bacterial promoter system provides tightly repressed gene expression in the absence of the inducer arabinose and highly derepressed gene expression in the presence of the inducer arabinose is the araB promoter of the Enterobacteriaceae family (U.S. Pat. No. 5,028,530 to Lai et al. and U.S. Pat. No. 6,803,210 to Better). As such, the transcription of the EGS may be regulated by arabinose when placed under the control of the arabinose operon.

A recently developed inducible promoter based on the Pseudomonas putida F1 can also be used as a regulatory element in the EGS, and is commercially available from Q-Biogene. The regulatory gene CymR controls the conversion of p-cymene to p-cumate in Pseudomonas putida F1, as well as the degradation of cumate. The regulatory element may encompass a gene encoding CymR and the cym operon. When cumate is not present, CymR may bind the operon and therefore the promoter is blocked. When cumate is present, it may bind cumate and the operon can now function. The cym operon can be linked to the EGS and expression may be induced when cumate is added to a host cell containing the EGS and cym operon, or administered to a transgenic animal with the EGS.

The regulatory element can also be induced by the messenger cyclic adenosine monophosphate (cAMP). Transcription factors are activated by cAMP acting through cAMP-responsive elements (CREs) found in various gene promoters. In addition to cAMP, CRE can be activated by other signalling pathways. Promoters containing one or multiple CREs can thus be used to control the expression of a gene (U.S. Pat. No. 6,596,508 to Durocher). In the present disclosure, the cAMP inducible promoter may be placed upstream and therefore control the expression of EGS. cAMP analogues can be used to induce the expression of the heterologous sequence (Schwede et al., Biochemistry, 39:8803-8812 (2000)).

In other embodiments of the present disclosure, the regulatory factor is a ligand for steroid receptors. Ligands for the steroid receptors can be produced in nature or synthetically. Steroid receptors are generally intracellular receptors and become activated when it binds its ligand. The ligand-binding domain of the receptor typically provides the means by which the 5′ regulatory region of the target gene is activated in response to the hormone. The DNA-binding domain comprises a sequence of amino acids that binds to a hormone response element (HRE). A response element is generally located in the 5′ regulatory region of a target gene that is activated by the hormone. The transactivation domain typically comprises one or more amino acid sequences acting as subdomains to affect the operation of transcription factors. Binding of the ligand generally causes a conformational change in the receptor and allows the transactivation domain to affect transcription of the coding sequence in the target gene, resulting in production of the target. Inducible promoters have been designed in which the ligand-binding domain (LBD) is linked to the target sequence. Promoters have also been designed in which the HRE is integrated into the promoter. An example is the glucocorticoid response element (GRE). It has been adapted into a synthetic promoter designed to be responsive to a number of steroid receptors other than the glucocorticoid receptor, such as the progesterone, androgen, and minearlocorticoid receptor (U.S. Pat. No. 5,512,483 to Mader et al.). Also, LBD of different steroid receptors can be combined with DNA binding domains of different steroid receptors (U.S. Pat. No. 4,981,784 to Evans et al.). Other receptors and their ligands have been used to control gene expression successfully in mammalian cells (U.S. Pat. No. 5,534,418 to Evans et al.). Such methods may be used in the present disclosure to control the expression, and therefore, activity of the EGS.

Steroid receptors that can be used to derive regulatory components from may include the vitamin A receptor, vitamin D receptor, retinoid receptor, or thyroid hormone receptor. In preferred embodiments, the regulatory elements are derived from the estrogen receptor (ER), glucocorticoid receptor (GR), mineralocorticoid receptor (MR), androgen receptor (AR), or progesterone receptor (PR). Ligands for the steroid receptors include its natural ligand and all its derivatives and analogues. Ligands may include vitamin A, retinoic acid, tretinoin, vitamin D1, D2, D3, D4 and D5, and most preferably are hormones such as thyroid hormones, estrogen, glucocorticoids, progesterone, androgen, and mineralocorticoids, their derivatives and analogues. Thyroid hormones include thyroxine (T4) and triiodothyronine (T3) and the synthetic levothryoxine. Synthetic glucocorticoids to induce the regulatory element can include hydrocortisone, cortisol acetate, prednisone, prednisolone, methylprednisolone, dexamethasone, betamethasone, triamcinolone, beclometasone, fludrocortisone, aldosterone, and deoxycorticosterone acetate (DOCA). Progesterone is widely available in commercial forms, such as the products Prometrium, Utrogestan and Microgest, as are progestins such as norethynodrel, norethindrone, norgestimate, norgestrel, levonrgestrel, medroxyprogesterone, and desogestrel. These may also be used as regulatory factors. Androgens such as testosterone, dehydroepiandrosterone, androstendione, androstenediol, androsterone, and dihydrotestosterone may also be regulatory factors. Other regulatory factors can include fludrocortisone acetate.

Another exemplary regulatory element is an ecdysone-inducible system. Ecdysones are insect steroidal hormones and are in use as an inducible element (WO 96/27673 to CIBA-GEIGY AG; U.S. Pat. No. 5,514,578 to Hogness et al.; WO 96/37609 to Zeneca Ltd; WO 93/03162 to Genentech, Inc). Ecdysone, the generic term frequently used as an abbreviation for 20-hydroxyecdysone, controls timing of development in many insects. Generally, ecdysone triggers changes in tissue development that results in metamorphosis. The Ecdysone receptor (EcR) binds to ecdysone and transactivates gene expression of a target gene in the nucleus. Other chemicals, such as the non-steroidal ecdysone agonist RH5849 (Wing, Science 241:467 469 (1988)), may also act as a chemical ligand for the ligand-binding domain of EcR. In preferred embodiments, the regulatory element is a promoter under the control of an ecdysone inducer, and the regulatory element controls the expression of EGS. The promoter can be induced by ecdysone or any of its analogues such as ponasterone A, and muristerone A. EGS can be linked to a pol III promoter that is under the control of ponasterone A, an ecdysone inducer (Kovrigina et al, RNA 11:1588-1595 (2005)).

In some embodiments, the regulatory element is a regulatable ribozyme. The regulatable ribozyme can be linked to the EGS, or independent of the EGS. Ribozymes are defined as RNA molecules having enzyme like activity. All naturally occurring ribozymes known to date, with the exception of RNAase P, work in cis and is engineered to work in trans, i.e., on another molecule. Regulatable ribozymes can be constructed as described in U.S. Pat. No. 5,741,679 to George et al. The ribozyme sequence may be linked to a ligand-binding sequence, placing the activity of the ribozyme under the control of that ligand and requiring the presence of the ligand for activation or inactivation, thus affecting the binding to or cleavage of the target nucleic acid. The ligand may be selected from the group consisting of nucleic acid molecules, proteins, polysaccharides, sugars, organic molecules and inorganic molecules. The ribozyme may be derived from a ribozyme selected from the group consisting of hammerhead ribozymes, axehead ribozymes, newt satellite ribozymes, Tetrahymena ribozymes, and RNAase P.

The regulatory element can also be based on the Cre-Lox system. The Cre-Lox system is well known in the art (Sternberg and Hamilton, J Mol Biol 150:467-486 (1981); Sauer and Henderson, PNAS 85:5166-5170 (1988)). It has been applied to yeast, plants, mammalian cell cultures, and mice (Araki et al., J Biochem (Tokyo) 122:977-982 (1997)). The system begins with the Cre protein, a site-specific DNA recombinase. Cre can catalyse the recombination of DNA between specific sites in a DNA molecule. These sites, known as loxP sequences, contain specific binding sites for Cre that surround a directional core sequence where recombination can occur. When cells that have loxP sites in their genome express Cre, a reciprocal recombination event will occur between the loxP sites. The double stranded DNA is cut at both loxP sites by the Cre protein and then ligated. The FLP-FRT system is similar to the Cre-lox system. It involves using flippase (FLP) recombinase, derived from the yeast Saccharomyces cerevisiae (Sadowski, Prog Nucleic Acid Res Mol Biol 51:53-91 (1995)). FLP recognizes a pair of FLP recombinase target (FRT) sequences that flank a genomic region of interest. The Cre-Lox system can be combined with an inducible promoter and within the EGS may be loxP sites. Expression of the Cre recombinase may be dependent on the inducible promoter, such that when the promoter is activated, Cre recombinase is expressed, act on the loxP sites, and inactivate the EGS. The loxP sites can also flank the EGS if EGS is linked to a promoter such that its expression is dependent on being linked to the promoter. The analogous FLP-FRT system can also be used instead of the Cre-Lox system.

Also, tissue specific regulation can also be achieved by using a regulatory element. The regulatory element can be designed to be inducible only in a specific tissue. For example, if the EGS is to be expressed only in the T-cell lineage, the Lck promoter may be used as the regulatory element and EGS expression may be dependent on the Lck promoter. If the EGS is not to express in the T-cell lineage, one example would be to use the Cre-Lox system, and have the Cre recombinase under the control of the Lck promoter such that Cre may be expressed only in T-cells and inactivates the EGS. The Lck promoter can be made inducible such that it will inactivate the EGS when a regulatory factor is applied. A number of other tissues specific promoters are known in the art. An example of a source of tissue specific promoters is TiProD (the Tissue-specific Promoter Database). It is a database of human promoter sequences for which some functional features are known. One can retrieve sets of promoters according to their tissue-specific activity. The database is accessible at http://tiprod.cbi.pku.edu.cn:8080/index.html.

In some embodiments, the regulatory factor may inhibit expression of the EGS by preventing the hybridization of the EGS to its target. For example, antisense oligonucleotides complementary to EGS may be regulatory factors. In one embodiment, after a number of rounds of EGS-mediated targeting of RNA, wherein the EGS may be recycled and re-used by the cell. Addition of antisense oligonucleotides that are complementary to the single-stranded EGS may inhibit further targeting by the EGS.

In another aspect of the present disclosure, the EGS may comprise a subcellular localization sequence, alone or in combination with a regulatory element. In some embodiments, the subcellular localization sequence and EGS may be regulated by a regulatory element. In other embodiments, only the EGS is regulated by a regulatory element. Alternatively, only the subcellular localization sequence is under the control a regulatory element. For example, the subcellular localization sequence may be under the Cre-lox system. The subcellular localization sequence may be flanked by loxP sites, and in the presence of Cre, the sequence cleaved.

A wealth of information on the structure of various subcellular localization sequences is known in the art. For instance, the signal sequences typically correspond to the first 5 to 30 amino acids present at the N-termini of virtually all nascent, secreted proteins and cell surface receptors. The signal sequence is typically cleaved from the protein upon translocation across the membrane. Additionally, the transmembrane domain that anchors a protein to the cell membrane generally comprises hydrophobic amino acid residues. The nuclear localization sequence typically comprises a stretch of basic amino acids. Other membrane-localization sequence including ER retention sequence, myristoylation, palmitation, and farnesylation sites are also well characterized (Nilsson et al. Cell 58:707-718 (1989); Mineo et al. J. of Biol. Chem. 272 (16) 10345-10348 (1997); Lee et al. J. of Cell Biol. 118 (5):1057-1070 (1992)). For example, there are publicly available databases with nuclear localization sequences and computer programs that predict potential nuclear localization sequences such as http://cubic.bioc.columbia.edu/predictNLS/ and http://psort.nibb.ac.jp/. Based on these and other studies, a skilled artisan can routinely identify and modify subcellular localization sequences of genes to target EGS to subcellular compartment of interest. In preferred embodiments, EGS is targeted to the nucleus or mitochondria.

The EGS may be targeted to the nucleus with nuclear localization sequences that are amino acid sequences, such as a basic amino acid sequence, or an amphipathic peptidem such as an amphipathic α-helical peptide. Nuclear localization elements may be oligonucleotide sequences that target the EGS to the nucleus. In preferred embodiments the nuclear localization elements are hexamer sequences, such as that described in Hwang et al., Science 315:97-100 (2007). The EGS may be linked to the hexamer sequence AGNGUN, where N is any nucleotide.

In other embodiments, the EGS is targeted to the mitochondria with mitochondrial localization sequences. The mitochondrial localization sequence may be a peptide or an oligonucleotide sequence. Mitochondrial targeting signal peptides tend to form amphiphilic helices. Elements important for mitochondrial targeting were recently identified in these helices. Arginine residues located on the hydrophilic side of such a helix may interact with corresponding negative charges on the surface of TOM22, a component of the mitochondrial protein import machinery. Hydrophobic Leu residues on the other side of the amphiphilic helix may be important for the interaction with TOM20, another component of the mitochondrial protein import machinery. One of ordinary skill in the art can modify mitochondrial localization sequences to localize EGS to the mitochondria.

In another aspect, the present disclosure provides a host cell comprising the EGS targeting a miRNA or mitochondrial mRNA. In some embodiments the host cell comprises a vector comprising the EGS. The cell may be a prokaryotic or eukaryotic cell type. The cells may be used for in vitro and in vivo assays. Cells include bacterial cells, mammalian cell lines, and cells in animals, such as mice, birds, dogs, cats, and humans. Cells may be transfected with the EGS and other relevant control molecules, techniques well known to one of ordinary skill in the arts. Preferably, cell lines are human epithelial and lymphoblastoid cell lines. A number of characterized human cell lines are available from the ATCC (American Type Culture Collection). Exemplary cell lines include human embryonic cell lines (Kovrigina et al, RNA 11:1588-1595 (2005)), C127 mouse cells (Plehn-Dujowich and Altman, PNAS 95: 7327-7332 (1998)), human T24 bladder carcinoma (Ma et al., Nature Biotech. 18:58-61 (2000)), B-lymphoblastoid cell lines m12-4-1 murine, Ramos B-lymphoblastoid cell lines, and 45/2w11 (Dreyfus et al., International Immunopharmacology 4:1013-1027 (2006)), human Jurkat T-lymphoblastoid and other lymphoblastoid cell lines.

Gene expression in a host cell may change when the host cell comprises, or is in contact with, EGS. Changes in gene expression may be determined by comparing control host cells without EGS, or with control EGS oligonucleotides, to host cells with EGS. Gene expression may be modulated by contacting the host cell with the EGS designed to target miRNA or mitochondrial mRNA. By targeting miRNA with EGS, miRNA may be cleaved by RNaseP, and as a result the gene expression of the miRNA targets may increase. EGS targeting of mitochondrial mRNA for RNaseP mediated cleavage may cause a decrease in the level of the gene in which the mitochondrial mRNA derived from. For example, the mRNA level and/or protein level of the mitochondrial gene may be decreased. Gene expression in a host cell may be modulated by more than one EGS. For example, a host cell may be in contact with two EGS, wherein one targets one miRNA and the other EGS targets another miRNA. In another embodiment, the host cell may comprise EGS targeting miRNA and mitochondrial mRNA. In further variations, EGS targeting other RNA species may be combined with EGS targeting miRNA and/or mitochondrial mRNA, such as EGS targeting mRNA. Modulation of gene expression may also be inducible, for example, by contacting the host cell with an EGS under the control of a regulatory element. EGS expression and activity may then be regulated, or induced, by a regulatory factor.

Synergy between antisense DNA, siRNA, aptamers, and EGS may permit multiple miRNA or mitochondrial mRNA targeting or increased miRNA elimination. For example, modulation of gene expression by EGS may be combined with antisense DNA oligonucleotides, siRNA and/or aptamers. The same or different miRNA may be targeted by EGS, siRNA, aptamers, and/or antisense DNA. Antisense DNA may bind pre-miRNA or pri-miRNA, preventing further miRNA processing, or may bind the miRNA precursors or mature miRNA to induce degradation of the hybrid by RNaseH. Antisense DNA oligonucleotides may be of any suitable length, from about 10 to 60 nucleotides in length, depending on the particular target. Antisense DNA oligonucleotides about 10 to 36 nucleotides long are preferred, and in particular embodiments, about 12 or about 21 nucleotides long.

Standard methods in the design of siRNA are known in the art (Elbashir et al., Methods 26:199-213 (2002)). In general, a suitable siRNA is between about 10-50, or about 20-25 nucleotides, or about 20-22 nucleotides. The target site typically has an AA dinucleotide at the 3′ end of the sequence, as siRNA with a UU overhang can be more effective in gene silencing. The remaining nucleotides generally exhibit homology to the nucleotides 3′ of the AA dinucleotides. In general, the siRNA typically exhibits at least about 50% homology to the target sequence, preferably at least about 70%, about 80%, 90% or even 95% homology to the target sequence. Where desired, potential target sites are also compared to the appropriate genome database, such that target sequences may have fewer than 70%, 60%, 50%, 40%, 30%, 20%, 10%, 5%, or even 1% homology to other genes. The readily available public database on the NCBI server, www.ncbi.nlm.nih.gov/BLAST is an example of a tool used to determine sequence homology. A public siRNA design tool is also readily available from the Whitehead Institute of Biomedical Research at MIT, http://jura.wi.mit.edu/pubint/http://iona.wi.mit.edu/siRNAext/.

The siRNA can also take the form of a hairpin siRNA. One may vary a number of known factors in designing a suitable siRNA. Such variables include the selection of siRNA target sequence, the length of the inverted repeats that encode the stem of a putative hairpin, the order of the inverted repeats, the length and composition of the spacer sequence that encodes the loop of the hairpin, and the presence or absence of 5′-overhang can vary depending on the target and the predicted inverted region; all of which can be varied or customized according to standard procedures in the art. The stem can be 19 to 20 nucleotides long, preferably about 19, 21, 25, or 29 nucleotides long. The loop can range from 3 nucleotides to 23 nucleotides, with preference for loop sizes of about 3, 4, 5, 6, 7 and 9 nucleotides. Databases available to the public to aid in the selection and design of hairpin siRNA are also available, such as www.RNAinterference.org, and online design tools, for both hairpin siRNA and siRNA are available from commercial sites such as Promega and Ambion.

Aptamers may be oligonucleotides, e.g. DNA or RNA. Aptamers may be created by selection of sequences from large random sequence pools, and may also exist naturally. Aptamers may be generated by methods known in the art or sequences obtained from a public database such as http://aptamer.icmb.utexas.edu. Aptamers may also bepeptides that bind a specific target. For example, they may be designed to inhibit protein interactions within a cell. They may comprise of a variable peptide loop attached at both ends to a protein scaffold. This typically allows the peptide aptamer to bind with greater affinity than an antibody. In preferred embodiments, the variable loop consists of at least 10 amino acids, preferably between 10 to 20 amino acids. The scaffold protein is generally a protein with good solubility, such as the bacterial protein thioredoxin-A. Peptide aptamers may be selected by any system, such as yeast two-hybrid.

The present disclosure also provides host cells comprising the subject EGS designed to target miRNA and mitochondrial mRNA. Another aspect of the present disclosure is transgenic animals comprising EGS targeting miRNA or mitochondrial mRNA. In some aspects, the transgenic animal may comprise a vector, or host cell, that comprises EGS targeting miRNA or mitochondrial mRNA. The transgenic animal may comprise EGS targeting miRNA and mitochondrial mRNA, as well as other RNA species. Transgenic animals may include mammals such as mice, rats, rabbits, dogs, or cats. Other mammals may include bovines, equines, or ovines.

MicroRNA (miRNA)

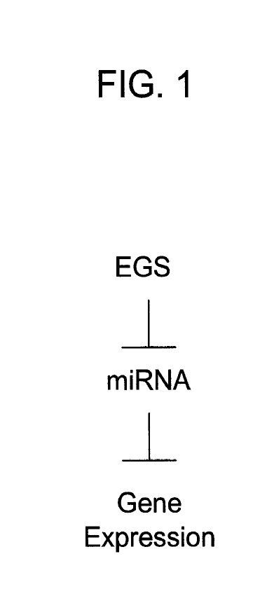

In one aspect of the present disclosure, EGS is designed to target microRNAs (miRNA) (FIG. 1). MiRNA negatively regulates partially complementary target mRNA and are typically 21-23 nucleotides in length. Recent work suggests two mechanisms of miRNA regulation of gene expression. One mechanism is by translation inhibition, where the target site for miRNA is in the 3′-UTR of the gene, and there is partial complementation between the miRNA and 3′UTR. The second mechanism is believed to be through near-perfect complementary sequences, and the target may be in the 3′UTR or the coding region. The second mechanism of miRNA is similar to that of siRNA, where the miRNA-mRNA complex goes through RNAi. As both mechanisms rely on partial base-paring with mRNA, miRNA target sites have been difficult to determine through bioinformatics. However, recent work has provided insight into the mechanism and as well as target sites. Current work suggests that perfect base-pairing of approximately 8 nucleotides between the 5′-end of miRNA and the target mRNA is critical, and 3′ end pairing may also modulate the activity of the miRNA. Target sites may also be flanked by adenosines (Brennecke et al., PloS Biol. 3:e85 (2005); Krek et al., Nat. Genet. 37:495-500 (2005); Lewis et al., Cell 120:15-20 (2005)). A single gene may be regulated by multiple miRNAs, and miRNA regulation may act synergistically. A single miRNA is also believed to be able to regulate multiple mRNAs, for example, some estimates have suggested a single human miRNA may regulate up to 100-200 genes. It has also been recently described that up to 30% of human genes are targets for miRNA regulation. As mentioned above, as miRNAs are discovered, many are entered into publicly available databases, such as http://www.microrna.org/ and http://microrna.sanger.ac.uk/.

MiRNA has been shown to be involved in a number of cellular processes, such as apoptosis, fat metabolism, development, differentiation, proliferation, immune response, and stress response. For example, miR-14 has been shown to have a role in fat metabolism in Drosophila. Other studies have shown that mice without miR-155 do not respond well to vaccination and are unable to develop immunity. Another miRNA, miR-181, was shown to direct the differentiation of human B cells (Chen et al., Science 303: 83-86 (2004)), while miR-373 regulates insulin secretion (Poy et al., Nature 432:226-230 (2004)), and other miRNAs regulate viral infections (Lecellier, C. H., et al., Science 308:557-560 (2005); Sullivan et al., Nature 435:682-686 (2005)).

Recent work has also shown a role for miRNA in diseases, such as cancer, immune diseases, neurological disease, developmental disease, as well as in viral infections. EGS may be designed to target miRNA, pri-mRNA, pre-miRNA, or mature forms of miRNA, resulting in upregulation of the cellular target the miRNA regulates. Design of EGS taking the form of antisense oligonucleotide targeting RNA may involve knowledge of the miRNA sequence of a cellular target. Recent work has sequenced over 250 small RNA libraries from different organ systems and cell types of humans and rodents (Landgraf et al., Cell 129.1401-1414 (2007)) and databases of miRNA sequences are also publicly available at sites such as http://www.microrna.org/ and http://microrna.sanger.ac.uk/. To minimize off-target effects, sufficient sequence homology of the EGS to the miRNA target is generally desired. In preferred embodiments, EGS is designed to target the pri-mRNA, pre-miRNA, or mature forms of miRNA, wherein overexpression of the miRNA is associated with diseases. In other embodiments, the miRNA to be targeted is miRNA that binds a gene for which underexpression is associated with disease. By targeting these miRNA, the results will likely be relief of symptoms of the disease or disorder.

Cancers such as chronic lymphocytic leukemia, pediatric Burkitt's lymphoma, gastric cancer, lung cancer, prostate cancer, and large cell lymphoma have been correlated with defects in miRNA expression. For example, two types of miRNA regulated by the c-Myc, a transcription factor implicated in some cancers, were shown to inhibit E2F1, a protein that regulates cell proliferation (O'Donnell et al., Nature 435:839-843 (2005)). Proteins of the argonaute/PAZ/PIWI family, which are components of both RISC and miRNPs, which is a class of ribonucleoproteins containing numerous miRNAs, have also been correlated with disease. It has been suggested that genes encoding these proteins are associated with cancer, for example, human Ago3, Ago1 and Ago4 reside in region 1p34-35, which is often lost in Wilms' tumors, and Hiwi, is located on chromosome 12q24.33, which has been correlated to the development of testicular germ cell tumors (Carmen et al., Genes Dev 16:2733-2742 (2002)). In addition, DICER, the enzyme which processes miRNAs and siRNAs, is poorly expressed in lung cancers (Karube et al., Cancer Sci. 96:111-115 (2005)).

In one embodiment, the EGS targets miRNA that are upregulated in tumor cells, for example in lung tumor cells, where miR-21 is upregulated. Without being bound by theory, it is believed that miR-21 targets the tumor suppressor TPM1, and may provide an explanation of the correlation between overexpression of miR-21 and tumor occurence (Zhu et al., J. Biol. Chem. 282:14328-14336 (2007)). In another example, miR-155 may be targeted by EGS, as miR-155 is believed to be overexpressed in diffuse large B cell lymphoma (DLBCL), or in Burkitt's Lymphoma (E is et al., Proc. Natl. Acad. Sci. U.S.A. 102:3627-3632 (2005); Metzler et al., Genes Chromosom. Cancer 39:167-1.69 (2004)). Overexpression of miR-155 has also been linked to oncogenesis and upregulation of multiple oncogene transcripts in malignant lymphoma cells (Costinean et al., Proc. Natl. Acad. Sci. USA 103:7024-7029 (2006)). Clusters of miRNAs have also been implicated in cancer. For example, the miR-17-92 polycistron is located in a region of DNA that is amplified in human B-cell lymphomas. It was discovered that the levels of the primary or mature microRNAs derived from the miR-17-92 locus are often substantially increased in these cancers (He et al., Nature 435:828-833 (2005)). These studies implicate the miR-17-92 cluster as a potential human oncogene. Another cluster, miR-106-363, has recently been implicated in T-cell leukemia (Landais et al. Cancer Res. 67:5699-707 (2007)).

Another group of miRNA, miR-15/16/195 may also have a role in cancer. Targets of miR-15/16/195 are believed to have roles in stimulating cell growth and possible roles in cancer development, for example, FGF2, CCND2, CCND1, CCNE1, and TGIF2. As another example, the miR-17/20/106, miR-19, and miR-25/32/95 families, based on predicted targets as described in US Pat. Application 2006/0185027, are believed to be involved in promoting growth and proliferation, and may also have a role in cancer. For example, transcription factor E2F1 may be a target of miR-20 and miR-106. As overexpression of E2F1 may lead to apoptosis, targeting miR-20 and/or miR-106 using EGS may result in cellular apoptosis, useful for treating cancerous cells.

A number of other miRNAs thought to be involved in cancer may also serve as targets for the EGS of the present disclosure. For example, miR-101 or miR-202 may also be targets for EGS. By reducing miR-101 or miR-202, N-MYC expression may be amplified resulting in growth inhibition or apoptosis. In another embodiment, miR-216 may be targeted by EGS, leading to overexpression of YB-1, resulting in a lowering of androgen, useful in treating prostate cancer cells. Another target for the EGS may be miR-138, as its target, FKHL7 may act as a tumor suppressor. EZF (Kruppel-like factor 4) may also be modulated by targeting miR-7. In another embodiment, the EGS may target miR-19a or miR-19b, as it may lead to increase of PTEN expression as loss of PTEN expression has been correlated to shortened survival in patients having melanoma and other types of cancer. Targeting of miR-19a may also increase CIS-6 expression levels, which is decreased in certain forms of cancer, such as breast cancer.

Other putative targets for EGS include miR-103, miR-107, miR-145, mIR-203, miR-124a, miR-221, miR-222, miR-124a, miR-34, miR-25, miR-92, miR-24, miR-143, miR-22, miR-125a, miR-125b, let-7a, miR-124a, miR-128, miR-135b, miR-19a, miR-1, miR-219, miR-23a, miR-26a, miR-27a, miR-29b, miR-138, or miR-34, wherein the respective targets are believed to be HMG-I (or HMG-Y), FLI-1, IRF-1, STAT3, SDF-1, ANG-1, HN¹, ERK (e.g., ERK4), proprotein convertase subtilisin-kexin type 7 precursor, Sema, Naked Cuticle Homolog 1, Homeodomain Interacting Protein Kinase 3 (HIPK3),. HIPK3, Mnt, Checkpoint Suppressor 1 (CHES1), SOCS-5, Dead-box Protein p68, Dead Ringer-Like 2, POU Domain Class 4, POU Domain Class 4, SMADI, Pim-1, nPKC delta, DAP-5, ETS Factor 3, MT3A, DNMT3A, Rhotekin, and NOTCH 1, each thought to have a role in cancer, for example, FKHL7 in endometrial or ovarian cancer, FLI-1 in Ewing's sarcoma, HMG-1 or HMG-Y in pancreatic cancer, EZF in gastric cancer, and ERK in lymphomas. Other miRNAs overexpressed in cancers may be identified by performing miRNA expression profiles of human cancers compared to control groups (Mak et al., Nature 435:834-838 (2005)). These miRNA may also be targets of the EGS. Other miRNA targets for EGS may include miRNA that are expressed in human prostate cancers, such as miR-100, miR-125b, miR-141, miR-143, miR-205, or miR-296 (Mitchell et al., Proc. Natl. Acad. Sci. USA 105:10513-10518 (2008)). The miRNA targets may be secreted by cells, such as miRNAs that are secreted by tumor cells, such as miR-141.

MiRNA also has a key role in the mammalian immune system. For example, miR-155 has a role in regulating T helper cell differentiation and the germinal center reaction to produce an optimal T cell-dependent antibody response. (That et al., Science 316:604-608 (2007)). Elimination of miR-155 has been linked to immunosuppression (That et al., Science 316:604-608 (2007), Rodriguez et al., Science 316:608-611 (2007). It is thought that miR-155 exerts this control, at least in part, by regulating cytokine production. Another miRNA, miR-150, is highly upregulated during the development of mature T and B cells. This suggests miR-150 most likely downregulates mRNAs that are important for pre- and pro-B cell formation or function, and its ectopic expression in these cells block further development of B cells (Zhou et al., Proc. Natl. Acad. Sci. USA 104:7080-7085 (2007)). Thus, another aspect of the present disclosure is the targeting of miRNAs involved in the immune system, such as miR-155 and miR-150, by EGS to modulate the immune system. In preferred embodiments, the miRNAs targeted are those involved in immune system diseases, such as autoimmune diseases or inflammatory diseases or conditions.

Other miRNAs that may be targeted include miR-133 or miR-133b, which are thought to have a role in the development of the T-cell surface glycoprotein CD4 precursor. Another example is miR-125b, believed to modulate the expression of TPR Repeat Protein 7, a protein that may control development of immune system cells. Another gene, LIF, has been correlated to arthritis, and is also believed to be regulated by miR-125b, as well as miR125a. MiR-19a may also be targed by EGS, thereby modulating megalin, a gene implicated in autoimmune diseases, such as rheumatoid arthritis, systemic lupus erythematosus, Bechcet's disease, systemic sclerosis, and osteoarthritis. In other embodiments, miR-29b may be targeted by EGS, as miR-29b is believed to modulate expression of Tribbles Homolog 2. Overexpression of Tribbles Homolog 2 has been observed in some types of autoimmune disease, such as autoimmune uveitis. In yet another embodiment, let-7a may be targeted by EGS. Let-7a is believed to modulate collagen alpha 1 (I) chain precursor expression. Reduced collagen expression has been observed in arthritis, osteogenesis imperfecta, and similar indications, thus targeting of let-7a may result in overexpression of collagen alpha 1 (I) chain precursor to ameliorate the effects of arthritis and similar conditions. In some embodiments, miR-34 is targeted by EGS. VAMP-2 may be modulated by miR-34, and VAMP-2 is downregulated in insulin deficiency, for example in diabetes mellitus. By targeting miR-34, VAMP-2 may be overexpressed.

Other miRNA implicated in inflammatory diseases or conditions, which include but are not limited to, acute allergic reactions, development of atropic diseases, and exacerbations of existing atopic conditions, may also be targeted by the EGS. Non-limiting exemplary atopic conditions are eczema, allergic conjunctivitis, allergic rhinitis, food allergies, anaphylaxis, and asthma, may also be targeted by EGS. MiRNAs involved in the symptoms from airway diseases, such as, but are not limited to, chronic bronchitis, surfactant depletion, chronic obstructive pulmonary disease (COPD), pulmonary transplantation rejection, pulmonary infections, inhalation burns, Acute Respiratory Distress Syndrome (ARDS), infantile and pregnancy-related RDS, cystic fibrosis, pulmonary fibrosis, radiation pulmonitis, tonsilitis, emphysema, esophagitis, cancers afflicting the respiratory system either directly such as lung cancer, esophageal cancer, and the like, or indirectly by means of metastases, as well as, cancer, which either directly or by metastasis afflict the lung, may also be targeted by the EGS. MiRNAs involved in increased airway inflammation, airway hyperresponsiveness (AHR), epithelial necrosis, airway wall oedema, mononuclear and granulocytic infiltrates, bronchoalveolar lympthoid tissue hyperplasia, goblet cell metaplasia, difficulties of breathing, and bronchoconstriction may also be targeted by the EGS. In preferred embodiments, the EGS targets miRNAs overexpressed in inflammaotry diseases or conditions.

The miRNAs that may be targeted by EGS may include miRNAs that bind and target suppressors or inhibitors of cytokines implicated in atopic diseases such as IL-4 and IL-13, and transcriptions factors implicated in the differentiation of TH2-type lymphocytes such as c-Maf, NF-AT, NF-IL-6, AP-1, STAT-6, and GATA-3. In some embodiments, the cytokine may be targeted by EGS as well as the miRNA of the inhibitor of the cytokine. The miRNA of suppressors or inhibitors of genes involved in IgE synthesis such as CD40 and its receptor, may also be targeted by EGS. Other miRNAs that may be targeted by EGS include miRNAs that bind and target suppressors or inhibitors of he cd3 complement, p53, and NF-κB transcription factors p50 and p65. EGS may also target miRNAs that target suppressors or inhibitors of genes upregulated in asthma, such as the adenosine-1 receptor A1 (A1), as well as other genes such as DDE recombinases, such as RAG proteins (e.g. recombination activating gene 1 (RAG-1) and RAG-2), retroviral integrase, and herpes recombinase (Dreyfus, Ann. Allergy Asthma Immunol. 97:567-576 (2006)). Other recombinases with motifs as described by Dreyfus and Gelfand, as described in U.S. Pat. Nos. 5,959,074 and 6,187,584 may also be targeted. EGS may also target miRNAs that target suppressors or inhibitors of other genes essential for B and T cell development, other DDE recombinases, and cytokines and growth factors involved in tissue changes and remodeling, including, IL-10 and its receptor, TGFβ and EGF, and their respective receptors, TBR11, ALK1, ALK2, ALK5, and activin.

Another aspect of the present disclosure is the design of EGS targeting miRNAs involved in neurodegenerative diseases such as Alzheimer's, for example, by altering expression of a gene involved in neural regulation pathways. For example, BDNF decreases have been associated with late-stage Alzheimer's disease, and miR-1 or miR-206 may regulate BDNF levels. Thus, miR-1 and/or miR-206 may be targeted by EGS. In another embodiment, EGS may target miR-101, a miRNA that may regulate Ras-related protein RAP-1B, expression of which may cause neurite growth. Other targets for neurodegenerative diseases may include miR-218, miR-101, and miR-23a.

In another aspect of the present disclosure, EGS may target miRNA that is overexpressed in cardiovascular or skeletal disease. Cardiac and skeletal muscle-specific miRNAs, e.g. miR-1, miR-1-2, miR-133, miR-208, have been identified (van Rooij et al., Science 316:575-579 (2007); Chien, Nature 447:389-390 (2007); U.S. Pat. Application 20060246491). The miRNAs appear to have a role in the conductance of electrical signals, heart muscle contraction, heart growth and heart morphogenesis. For example, miR-208 may regulate stress dependent cardiac hypertrophy regulation (van Rooij et al., Science 316:575-579 (2007)).

MiRNA appear to be highly tissue specific, as 80% of conserved vertebrate miRNA expressed during embryonic development are tissue specific. The EGS may target miRNA that is enriched in particular tissues or in particular cell types, such as cardiac tissue as described above, or in pancreatic islet cells, whereby the EGS may be designed to target miR-375, a pancreatic islet-specific miRNA that has been shown to suppress glucose-induced insulin secretion (Poy et al., Nature 432:226-230, (2004)).

The EGS of the present disclosure may also target viral miRNA. It is believed that miRNA may regulate gene expression in viruses, such as members of Herepesviridae family. For example, the highly oncogenic Marek's disease virus type 1 (MDV-1) and MDV-2, both avian herpesvirus, has been shown to encode miRNAs (Burnside et al., J. Virol. 80:8778-8786 (2006); Yao et al., J. Virol. 81:7164-7170 (2007)). It was also proposed that the MDV miRNAs function to enable MDV pathogenesis and contribute to MDV-induced transformation. Thus, by targeting viral miRNA, one may ameliorate viral infections and/or symptoms. The oncogenic gammaherpesviruses Kaposi's sarcoma herpesvirus and Espstein-Barr virus may also encode miRNA. Other viral miRNA may be miRNA derived from rotavirus, parainfluenza virus, respiratory synctyial virus, herpes virus, Flavivirus, human immunodeficiency virus, hepatitis virus, human papillomavirus, Ebola virus, Rous sarcoma virus, human rhinovirus, and poliovirus. Of particular interest are miRNA derived from HIV-1, HIV-2, HSV-1, HSV-2, hepatitis A, hepatitis B, hepatitis C, hepatitis D, hepatitis E, hepatitis G, rotavirus A, rotavirus B, rotavirus C, avian influenza virus, and human influenza virus, wherein the influenza virus can be influenza A, influenza B, and/or influenza C. Exemplary serotypes include H1N1, H2N2, H3N2, H5N1, H7N7, H1N2, H9N2, H7N2, H7N3, or H10N7.

### Mitochondrial RNA

In yet another aspect of the present disclosure, EGS is designed to target mitochondrial RNA, in particular mitochondrial mRNA. Approximately 80% of mitochondrial DNA (mtDNA) codes for functional proteins and encodes a number of polypeptides that function in the electron transport chain, integral to providng ATP, defects in mtDNA often lead to serious cellular, and in turn, organismal, dysfunction. Often, mitochondrial diseases affect multiple organ systems, for example, the brain, muscle, nerve, heart, pancrease, eye, hearing, kidney, and gastrointestinal system. Symptoms of mitochondrial diseases may include stroke, seizures, dementia, ataxia, developmental delay, weakness, pain, fatigue, neuropathy, cariomyopathy, heart failure, heart block, arrhythmia, diabetes, pancreatitis, retinopathy, optic neuropathy, sensorineural deafness, renal failure, diarrhea, pseudo-obstruction, and dysmotiliy. Broad categories of mitochondrial diseases have been classified, such as progressive external opthamoplegia (PEO), diabetes mellitus and deafness (DAD), Leber hereditary optic neuropathy (LHON), mitochondrial encephalomyopathy, lactic acidosis, and stroke-like syndrome (MELAS), myoclonic epilepsy and ragged-red fibers (MERRF), Leigh Syndrome (subacute sclerosing encephalopathy), neuropathy, ataxia, retinitis pigmentosa, and ptosis (NARP), Kerns-Sayre Syndrome (KSS), and myoneurogenic gastrointestinal encephalopathy (MNGIE).

The present disclosure provides a means for targeting mitochondrial mRNA, wherein the EGS may target mitochondrial RNA that amelioriates the effects or symptoms due to defective mtDNA. For example, the EGS may target mitochondrial RNA derived from defective mtDNA.

### EGS Library

The present disclosure also provides a library of EGS comprising a plurality of EGS designed to target one or more RNA species, for example, mRNA, miRNA, or mitochondrial mRNA. The present disclosure further provides a method of contacting a host cell with a library of EGS comprising a plurality of oligonucleotides designed to target a plurality of miRNA, or mitochondrial mRNA, analyzing the gene expression profile of the host cell as compared to a control cell, and identifying if expression of one or more genes is modulated by the library. The one or more EGS from the library that modulates gene expression can then be identified. Alternatively, the one or more genes that are modulated by the EGS may also be identified. The methods may further comprise providing analysis or identification of the EGS or EGS target(s) to an individual. The data relating to analysis or identification of the EGS or EGS target(s) may be provided to an individual by transmission of the data over a network.

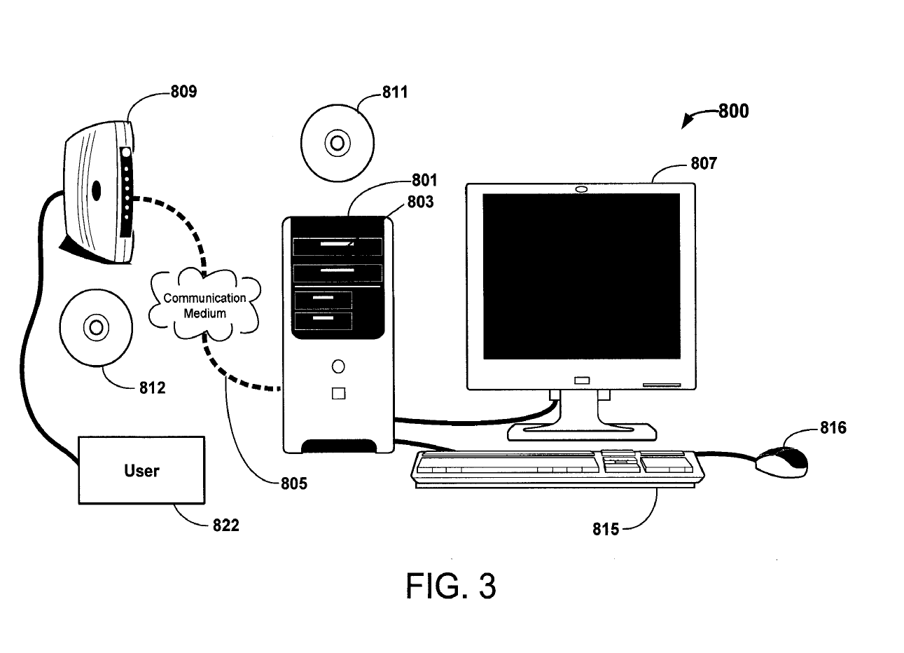

Accordingly, FIG. 3 is a block diagram showing a representative example logic device through which data relating to the analysis or identification of the EGS. FIG. 3 shows a computer system (or digital device) 800 to receive and store data, such as gene expression profiles cells contacted with or without an EGS library. The computer system may also perform analysis on the data, such as comparing expression profiles between cells contacted with or without an EGS library. The computer system 800 may be understood as a logical apparatus that can read instructions from media 811 and/or network port 805, which can optionally be connected to server 809 having fixed media 812. The system shown in FIG. 60 includes CPU 801, disk drives 803, optional input devices such as keyboard 815 and/or mouse 816 and optional monitor 807. Data communication can be achieved through the indicated communication medium to a server 809 at a local or a remote location.

The communication medium can include any means of transmitting and/or receiving data. For example, the communication medium can be a network connection, a wireless connection or an interne connection. Such a connection can provide for communication over the World Wide Web. It is envisioned that data relating to the present disclosure can be transmitted over such networks or connections for reception and/or review by a party 822. The receiving party 822 can be but is not limited to an individual. In one embodiment, a computer-readable medium includes a medium suitable for transmission of a result, such as identification of an EGS from an EGS library, of an analysis of expression profiles resulting from cells contacted with an EGS library.

In some embodiments, the library of EGS may comprise EGS targeting different RNA species, for example mRNA, miRNA, and/or mitochondrial mRNA. In another embodiment, the library of EGS may target a single RNA species, for example miRNA or mitochondrial mRNA. The library of EGS may target a specific miRNA, or a plurality of miRNA. In other embodiments, the library of EGS may target a specific mitochondrial mRNA, or a plurality of mitochondrial RNA.

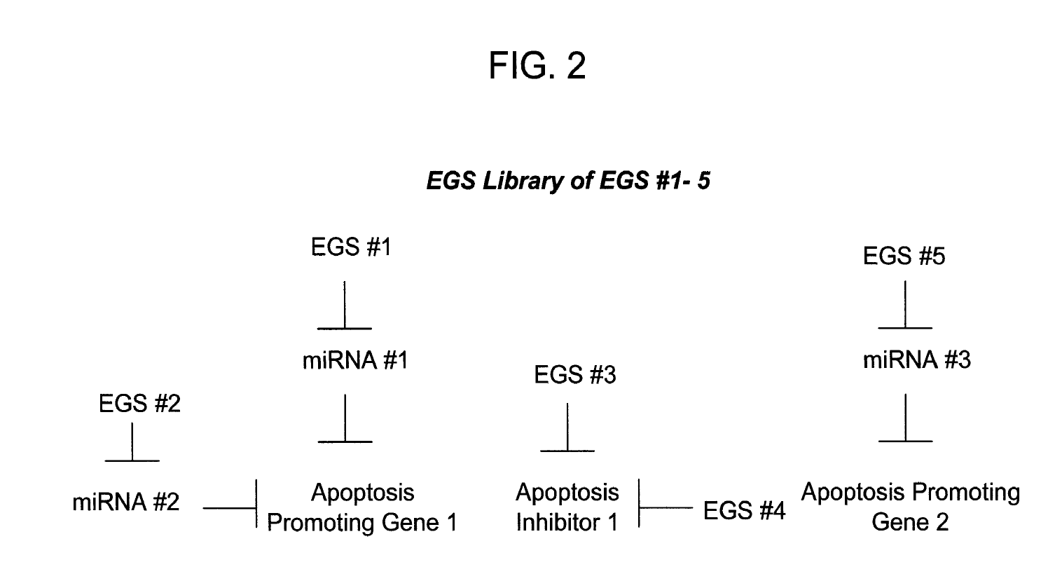

In another aspect of the disclosure, the EGS of the library may be designed to target a plurality of RNA species, wherein the RNA species affect a particular gene target. For example, the RNA species may be a plurality of miRNAs, wherein the miRNAs act on a single gene. In another embodiment, the RNA species may comprise different RNA species, such as miRNAs and mRNAs, wherein targeting of the miRNA and mRNAs enhance an effect of a gene. For example, targeting of a miRNA may enhance the expression of a gene, whereas targeting an mRNA of an inhibitor of the gene, will further enhance the expression of the gene, or the activity of the gene product. In other embodiments, the library of EGS may target a plurality of genes, wherein the genes have a common function in the cell, or affect a specific cellular process. For example, a group of genes may be involved in promoting apoptosis. The EGS may target miRNAs that inhibit apoptosis-promoting genes and/or mRNAs of inhibitors or suppressors of apoptosis, such as Akt, NK-κB, Mdm2, Bcl-2, Mcl-1, Bcl-w, Bcl-xL, and IAP (FIG. 2). In another embodiment, the EGS library may modulate an inflammatory response, for example, the TH2 response. Other cellular process may include, but are not limited to, cellular transformation, differentiation, or proliferation.

Cell Based Assays

The present disclosure also provides for cell-based assays with EGS designed to target miRNA or mitochondrial mRNA. In another aspect, cell-based assays are performed with a library of EGS. The EGS may be delivered to host cells by transfection. Transfection may utilize lipid carriers designed for experimental transfection of cells with nucleic acids. The EGS may be delivered via a vector containing a sequence which encodes and expresses EGS specific for a particular RNA. For example, small nuclease resistant EGS are readily taken up into T24 bladder carcinoma tissue culture cells with carrier lipids such as lipid transfection reagents Lipofectin or Lipofectase (Ma, et al., Antisense Nucleic Acid Drug Dev. 8:415-426 (1998)). Uptake of these EGS may be noted by using 5 fluoresceinated EGS and detected by confocal microscopy. Stability of the EGS may be determined by quantitative PCR using specific primers for the EGS and other techniques such as Northern blotting.

The gene expression profile of a host cell contacted by the library of EGS may be analyzed by detecting the amount of target gene mRNA (e.g. using real time PCR) or the amount of polypeptide encoded by the target gene mRNA (Western blot analysis) after contact with a library of EGS, or a particular EGS, in comparison to a control not in contact with the library, or particular, EGS. Gene chips are readily available (e.g. more than 20,000 expressed sequence tags and controls, Affymetrix, Santa Clara, Calif.), and specific gene chips, for example, inflammatory cytokines and receptors (OligoGEArray, Superarray Bioscience, Frederick Mass.), apoptosis and developmental genes (DualChip, Eppendorf) are also available. Custom DNA chips can also be designed (Affymetrix, Santa Clara, Calif.).

The RNA species targeted by the EGS may be detected. RNA levels may be detected after incubation with various EGS, various EGS libraries, or various amounts of EGS. The RNA levels after incubation may be detected by RT-PCR, Northern blotting, reporter systems, or by other means known in the art. In one embodiment, arrays may be used to detect RNA levels. MiRNA arrays, probes, and markers are readily available (Ambion, Austin, Tex.). MiRNA may also be detected by using LNA (locked nucleic acid) oligos, which generally have extremely high binding affinity (Tm˜±100 deg C. for 20 mer oligo), and can be used in ordinary in situ hybridizations of miRNA from whole mount or sectioned material. LNA arrays are also commercially available (Exiqon, Denmark). MiRNA may also be detected by “sensor” reporters. A reporter gene with 3′UTR and an engineered miRNA binding site may be used, such that binding of mMiRNA induces expression of the reporter, for example, GFP, and detection of GFP may be indicative of miRNA expression.

An in vitro cleavage assay can be used to measure the percentage of substrate RNA remaining after incubation with EGS. In one embodiment, the EGS and the presence of a non-limiting amount of RNAse P is used as an indicator of the potential activity of the EGS/RNAse P complex. EGS/RNAse P complexes that exhibit the highest in vitro activity may be selected for further testing. The percentage of RNA remaining can be plotted as a function of the EGS concentration. The catalytic efficiency of an EGS/RNAse P can be expressed as k_{cat}/Kₘ (where k_{cat} is the rate constant of cleavage and Kₘ is the Michaelis constant), the second order rate constant for the reaction of a free EGS and substrate RNA molecule. Following the methods of Heidenreich and Eckstein, J. Biol. Chem., 267:1904-1909 (1992), k_{cat}/Kₘ may be determined using the formula where F is the fraction of substrate left, t is the reaction time, and [C] is the EGS concentration.

Formulations

The EGS of the present disclosure can be formulated to for administration to animals, such as, but not limited to, mice, birds, dogs, cats, and humans. Pharmaceutical compositions of the EGS may comprise an effective amount of EGS, for example, an amount that modulates expression of a gene, in admixture with a pharmaceutically acceptable carrier, for example, an adjuvant/antigen presentation system such as alum. Other adjuvant/antigen presentation systems, for instance, MF59 (Chiron Corp.), QS-21 (Cambridge Biotech Corp.), 3-DMPL (3-Deacyl-Monophosphoryl Lipid A) (RibiImmunoChem Research, Inc.), clinical grade incomplete Freund's adjuvant (IFA), fusogenic liposomes, water soluble polymers or Iscoms (Immune stimulating complexes) may also be used. Other exemplary pharmaceutically acceptable carriers or solutions are aluminum hydroxide, saline and phosphate buffered saline. The EGS in admixture with a pharmaceutically acceptable carrier can be formulated with other components that modulates expression of a gene, such as siRNA, antisense oligonucleotides, aptamers, and the like.

In vitro studies demonstrate the use of modified EGS for targeted use in human diseases. It has been shown that small nuclease resistant EGS are readily taken up into T24 bladder carcinoma tissue culture cells with carrier lipids at a concentration of 1.mu.Molar EGS and 10.mu.Molar lipid transfection reagents Lipofectin or Lipofectase (Ma, et al., Antisense Nucleic Acid Drug Dev. 8:415-426 (1998)). Uptake of these EGS was noted in both cytoplasm and nuclei of nearly every cell using 5 fluoresceinated EGS detected by confocal microscopy. Significant decreases in targeted gene expression were demonstrated in this model in the absence of observed toxicity. The formulations may contain an effective amount of EGS to reach a final EGS concentration of 1 micromolar or less in pulmonary extra-cellular fluid (approximately 10-15 cc) to decrease levels of targeted mRNA for days or weeks following intranasal administration. For example, this range of EGS concentration can be achieved by intranasal instillation of 0.01 micromoles of EGS. Like conventional asthma medications it is anticipated that EGS can be shipped through the mail and stored at room temperature, but unlike conventional therapy it is expected that a single dose will have therapeutic effects for days or even weeks due to long term effects upon target protein synthesis (Ma, et al., Nat. Biotechnol. 18(1):58-61 (2000) and Ma, et al., Antisense Nucleic Acid Drug Dev. 8:415-426 (1998)).

Nyce et al. have shown that antisense oligodeoxynucleotides (ODNs) termed RASONS (Respirable Anti-Sense OligoNucleotide Sequences) when inhaled bind to endogenous surfactant (a lipid produced by lung cells) and are taken up by lung cells without a need for additional carrier lipids (Nyce and Metzger, Nature, 385:721-725 (1997)). These observations indicate that oligonucleotide therapy directed at the lung may have particularly favorable rational and could alter disease in the lungs without systemic effects through localized or targeted effects of the therapy to lung tissues.

For therapeutic purposes, a DNA vector encoding an EGS can be utilized, such as a plasmid DNA vector or retroviral vector. Methods for creating such vectors are well known to one of ordinary skill in the art (see for example, U.S. Pat. No. 5,869,248 to Yuan, et al., U.S. Pat. No. 5,728,521 to Yuan, et al., Zhang and Altman, J. Mol. Biol. 342:1077-1083 (2004); and Plehn-Dujowich and Altman, PNAS USA 95:7327-7332 (1998)).

The EGS may be administered topically, locally or systemically in a suitable pharmaceutical carrier. Remington's Pharmaceutical Sciences, 15th Edition by E. W. Martin (Mark Publishing Company, 1975), discloses typical carriers and methods of preparation. Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

The EGS may also be encapsulated in suitable biocompatible microcapsules, microparticles or microspheres formed of biodegradable or non-biodegradable polymers or proteins or liposomes for targeting to cells. Such systems are well known to those skilled in the art and may be optimized for use with the appropriate EGS. The formulations may also be encapsulated to protect the EGS against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are known to one of ordinary skill in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP 0043075.

Formulations for parenteral administration may include sterile aqueous solutions which may also contain buffers, liposomes, diluents and other suitable additives. For intramuscular, intraperitoneal, subcutaneous and intravenous use, the EGS containing formulations may generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present which might affect or mediate uptake of EGS in the cells that express the target gene. Such substances include, for example, micellar structures, such as liposomes or capsids. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In a preferred embodiment, the EGS is formulated for pulmonary delivery. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorbtion occurs. The alveoli are covered by a thin epithelium without cilia or a mucus blanket and secrete surfactant phospholipids (Patton and Platz. Adv. Drug Del. Rev. 8: 179-196 (1992)).

The term aerosol as used herein refers to any preparation of a fine mist of particles, which can be in solution or a suspension, whether or not it is produced using a propellant. Aerosols can be produced using standard techniques, such as ultrasonication or high pressure treatment.

Carriers for pulmonary formulations can be divided into those for dry powder formulations and for administration as solutions. Aerosols for the delivery of therapeutic agents to the respiratory tract have been developed. See, for example, Adjei and Garren, Pharm. Res., 7: 565-569 (1990); and Zanen and Lamm, Int. J. Pharm., 114: 111-115 (1995). For administration via the upper respiratory tract, the formulation can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers are typically physiologically compatible and include, simply by way of example, phosphate buffers. For example, a representative nasal decongestant is described as being buffered to a pH of about 6.2 (Remington's Pharmaceutical Sciences 16th edition, Ed. Arthur Osol, page 1445 (1980)). One skilled in the art can readily determine a suitable saline content and pH for an innocuous aqueous solution for nasal and/or upper respiratory administration.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent may be selected based on its ability to readily aerosolize the formulation. The solvent does not detrimentally react with the EGS. An appropriate solvent may be used that dissolves the EGS, or forms a suspension of the EGS, or components thereof. A suspension may also be referred to as a dispersion herein. The solvent moreover should be sufficiently volatile to enable formation of an aerosol of the solution or suspension. Additional solvents or aerosolizing agents, such as freons, can be added as desired to increase the volatility of the solution or suspension.

Dry lipid powders can be directly dispersed in ethanol because of their hydrophobic character. For lipids stored in organic solvents such as chloroform, the desired quantity of solution may be placed in a vial, and the chloroform evaporated under a stream of nitrogen to form a dry thin film on the surface of a glass vial. The film typically swells easily when reconstituted with ethanol. To fully disperse the lipid molecules in the organic solvent, the suspension may be sonicated. Nonaqueous suspensions of lipids can also be prepared in absolute ethanol using a reusable PARI LC Jet+ nebulizer (PARI Respiratory Equipment, Monterey, Calif.).

A number of pharmaceutical preparations for pulmonary delivery of drugs have been developed. For example, U.S. Pat. No. 5,230,884 to Evans et al. discloses the use of reverse micelles for pulmonary delivery of proteins and peptides. Reverse micelles are typically formed by adding a little water to a nonpolar solvent (e.g. hexane) to form microdroplets. In this medium, a surfactant (detergent) may orient itself with its polar heads inward, so that they are in contact with the water and the hydrophobic tails outward. The tiny droplets of water may be surrounded by surfactant, and the protein to be delivered dissolved in the aqueous phase.

U.S. Pat. No. 5,654,007 to Johnson et al. discloses methods for making an agglomerate composition containing a medicament powder (e.g. proteins, nucleic acids, peptides, etc.) wherein a nonaqueous solvent binding liquid (a fluorocarbon) is used to bind the fine particles into aggregated units. The agglomerate composition has a mean size ranging from 50 to 600 microns and is allegedly useful in pulmonary drug delivery by inhalation.

These materials can be used for delivery of formulation to the lungs, modified as necessary to deliver the correct dosage of surface modifying agent at a desired rate and to a preferred location within the lung.

Dry powder formulations (“DPFs”) with large particle size have improved flowability characteristics, such as less aggregation (Visser, Powder Technology 58: 1-10 (1989)), easier aerosolization, and potentially less phagocytosis. (Rudt and Muller, J. Controlled Release, 22:263-272 (1992); Tabata and Ikada, J. Biomed. Mater. Res., 22:837-858 (1988)). Dry powder aerosols for inhalation therapy are generally produced with mean diameters primarily in the range of less than 5 microns. (Ganderton, D., J. Biopharmaceutical Sciences, 3:101-105 (1992); and Gonda, I. “Physico-Chemical Principles in Aerosol Delivery,” in Topics in Pharmaceutical Sciences 1991, Crommelin, D. J. and K. K. Midha, Eds., Medpharm Scientific Publishers, Stuttgart, pp. 95-115, (1992)), although a preferred range is between one and ten microns in aerodynamic diameter. Large “carrier” particles (containing no drug) have been co-delivered with therapeutic aerosols to aid in achieving efficient aerosolization among other possible benefits. (French et al., J. Aerosol Sci., 27:769-783 (1996)). Another method of making fine dry particles is by forming a composition of the RIC, or components thereof, with a supercritical or near critical fluid (U.S. Pat. No. 6,630,121 to Sievers et al.).

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter, and tap density for localized delivery to selected regions of the respiratory tract such as the deep lung or upper airways. For example, higher density or larger particles may be used for upper airway delivery. Similarly, a mixture of different sized particles, provided with the same or different RIC, or any of its components, may be administered to target different regions of the lung in one administration.

Formulations for pulmonary delivery include unilamellar phospholipid vesicles, liposomes, or lipoprotein particles. Formulations and methods of making such formulations containing nucleic acid are well known to one of ordinary skill in the art. Liposomes may be formed from commercially available phospholipids supplied by a variety of vendors including Avanti Polar Lipids, Inc. (Birmingham, Ala.). The liposome-associated RIC, or any of its components, may be prepared by mixing a solution of the RIC, or any of its components, with reconstituted lipid vesicles. In one embodiment, the liposome can include a ligand molecule specific for a receptor on the surface of the target cell to direct the liposome to the target cell. Toxicity and therapeutic efficacy of such formulations can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

Administration

The subject EGS has a variety of applications. The subject EGS may provide a method for modulation gene expression in a subject. The EGS may be stable and readily prepared in large quantities resulting in cost savings and highly cost effective and easy to administer due to their unique chemical properties and mechanism of active. The cost of EGS is estimated to be minimal, thereby providing an inexpensive method to modulate gene expression.

In one embodiment, an effective amount of EGS is administered to a human patient in need of therapeutic or prophylactic treatment. The EGS formulation can be administered in a subject together, as a single pharmaceutical composition, or they may be administered substantially simultaneously, sequentially, at preset intervals throughout the day or treatment period, at different frequencies, or using the same or different routes of administration.

The formulations may be administered by any means known in the art including, but not limited to oral or parenteral routes, including intravenous, intramuscular, intraperitoneal, subcutaneous, transdermal, and airway (aerosol) administration. In preferred embodiments, the formulations are administered via inhalation or nasal application to the lung. The formulations are administered to a patient in need of treatment or prophylaxis. The formulations can be administered to animals or humans.

Previous studies demonstrated optimal targeting of EGS to be at a concentration of approximately 1 micromolar in pulmonary fluids in vitro (Ma, et al., Nat. Biotechnol. 18(1):58-61 (2000)). Dosage levels of the EGS is of the order of from about 0.1 mg to about 140 mg per kilogram of body weight per day are useful (about 0.5 mg to about 7 g per patient per day). The dosage can be about 0.1 mg, 0.5 mg, 1 mg, 5 mg, 10 mg, 20 mg, 30 mg, 50 mg, 100 mg, 120 mg or 140 mg per kilogram of body weight per day. Dosage unit forms of the EGS may generally contain between from about 1 mg to about 500 mg of an active ingredient. The EGS is preferably about 1 mg, 2 mg, 5 mg, 10 mg, 25 mg, 50 mg, 100 mg, 150 mg, 200 mg, 250 mg, 300 mg, 350 mg, 400 mg, 450 mg, or 500 mg. The amount of active ingredient that can be combined with the carrier materials to produce a single dosage form may vary depending upon a variety of factors including the activity of the specific compound employed, the age, body weight, general health, sex, diet, time of administration, route of administration, and rate of excretion, drug combination, and preexisting conditions.

The composition can be systemically administered, subcutaneously or intramuscularly, in the form of an acceptable subcutaneous or intramuscular solution. Inoculation can be effected by surface scarification or by inoculation of a body cavity. The preparation of such solutions, having due regard to pH, isotonicity, stability and the like is within the skill in the art. The dosage regimen may be determined by the attending physician considering various factors known to modify the action of drugs such as for example, physical condition, body weight, sex, diet, severity of the condition, time of administration and other clinical factors.

In addition to their administration individually or multiply, as discussed above, the EGS can be administered in combination with other known agents effective in treatment of diseases. In any event, the administering physician can adjust the amount and timing of the administration on the basis of results observed using standard measures of efficacy known in the art. The EGS can be used directly in combination with a pharmaceutically acceptable carrier to form a pharmaceutical composition suited for administrating to a patient. Alternatively, the EGS or its components can be delivered via a vector containing a sequence which encodes and expresses the EGS specific for a particular RNA.

Direct delivery involves the insertion of pre-synthesized heterologous sequences into the target cells, usually with the help of lipid complexes (liposomes) to facilitate the crossing of the cell membrane and other molecules, such as antibodies or other small ligands, to maximize targeting. Because of the sensitivity of RNA to degradation, in many instances, directly delivered heterologous sequences may be chemically modified, making them nuclease-resistant, as described above. This delivery methodology allows a more precise monitoring of the therapeutic dose.

Vector-mediated delivery generally involves the infection of the target cells with a self-replicating or a non-replicating system, such as a modified viral vector or a plasmid, which produces a large amount of the heterologous sequence encoded in a sequence carried on the vector. Targeting of the cells and the mechanism of entry may be provided by the virus, or, if a plasmid is being used, methods similar to the ones described for direct delivery of heterologous sequences can be used. Vector-mediated delivery may produce a sustained amount of heterologous sequences. It is typically substantially cheaper and generally requires less frequent administration than a direct delivery such as intravenous injection of the heterologous sequences. It is desirable that an effective amount of the heterologous sequence be delivered in a form which minimizes degradation of the heterologous sequence before it reaches the intended target site. Most preferably, the components of the EGS are within the same cell.

In preferred embodiments, the subject EGS is delivered by to the pulmonary system or other tissues, using topical inhaled transient or stable expression systems that may lead to regulated duration of therapy and tissue specific effects without systemic effects. Nuclear targeting of the heterologous sequence, for example, EGS with a hexamer targeting sequence, may also prevent activation of cytoplasmic Toll receptor response.

Pulmonary administration of therapeutic compositions comprised of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm.sup.3, porous endothelial basement membrane, and it is easily accessible. Therefore, intranasal delivery of complex molecules such as the RIC, or any of its components, may provide therapies for the reduction of pathological responses involved in induced immunity.

For pulmonary administration, formulations can be administered using a metered dose inhaler (“MDI”), a nebulizer, an aerosolizer, or using a dry powder inhaler. Suitable devices are commercially available and described in the literature. Inhaled aerosols have been used for the treatment of local lung disorders including asthma and cystic fibrosis (Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)). Considerable attention has been devoted to the design of therapeutic aerosol inhalers to improve the efficiency of inhalation therapies. (Timsina et. al., Int. J. Pharm., 101: 1-13 (1995); and Tansey, I. P., Spray Technol. Market, 4: 26-29 (1994)).

The formulation may be administered alone or in any appropriate pharmaceutical carrier for administration to the respiratory system. Delivery may be achieved by one of several methods. For example, the patient can mix a dried powder of the heterologous sequence with solvent and then nebulize it. It may be more appropriate to use a pre-nebulized solution, regulating the dosage administered and avoiding possible loss of suspension. After nebulization, it may be possible to pressurize the aerosol and have it administered through a metered dose inhaler (MDI). Nebulizers create a fine mist from a solution or suspension, which is inhaled by the patient. The devices described in U.S. Pat. No. 5,709,202 to Lloyd, et al., can be used. An MDI typically includes a pressurized canister having a meter valve, wherein the canister is filled with the solution or suspension and a propellant. The solvent itself may function as the propellant, or the formulation may be combined with a propellant, such as freon. The formulation may be a fine mist when released from the canister due to the release in pressure. The propellant and solvent may wholly or partially evaporate due to the decrease in pressure. Other devices that can aerosolize and/or deliver the RIC to the respiratory system are well known to one in the art (examples include, but not limited to, U.S. Pat. No. 4,735,217 to Gerth et al., U.S. Pat. No. 5,743,252 to Rubasmen, U.S. Pat. No. 6,546,929 to Burr et al., U.S. Pat. No. 6,234,167 to Cox et al. and U.S. Pat. No. 6,655,379 to Clark et al.). The formulation may be administered in other ways depending on whether local or systemic treatment is desired, and on the area to be treated. Administration may be topically, orally, by inhalation, or parenterally.

The formulation of the EGS may be administered in a single dose, followed by other doses given at subsequent time intervals required for a particular treatment. The EGS can also be administered in sub-doses, each sub-dose preferably correspondingly smaller in order to achieve the total daily dosage. The dosage unit can also be compounded for delivery over several days, e.g., using a conventional sustained release formulation which provides sustained release of the heterologous sequence over a several day period. Sustained release formulations are well known in the art. In this embodiment, the dosage unit contains a corresponding multiple of the daily dose.

The EGS could be administered on a weekly basis due to prolonged effects of the EGS upon expression and function. One of skill in the art will appreciate that certain factors may influence the dosage and timing required to effectively treat a subject, including but not limited to the severity of induced immune response, predisposing conditions, previous treatments, the general health and/or age of the subject, and other diseases present. Treatment of a subject with a therapeutically effective amount of a composition can include a single treatment or a series of treatments. The treatment can be varying dosages and number of treatments of the RIC or its components. Estimates of effective dosages and in vivo half-lives for the EGS can be made using conventional methodologies or on the basis of in vitro and in vivo testing using cell culture assays and appropriate animal models.

The data obtained from cell culture assays and animal studies can be used in formulation a range of dosage for use in humans. The dosage of compositions of the disclosure lies preferably within a range of circulating concentrations that include the ED50 with little or no toxicity. The dosage may vary within this range depending upon the dosage form employed and the route of administration utilized. For any EGS used in the method of the disclosure, the therapeutically effective dose can be estimated initially from cell culture assays. A dose may be formulated in animal models to achieve a circulating plasma concentration range of the EGS that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

There are a variety of mouse models for the study of various human diseases. Mouse repositories can be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of the EGS, as well as for determining a therapeutically effective dose. The EGS and appropriate controls can be instilled into the nasal passage of live mice as a model of efficacy and pharmacokinetics of the EGS in the reduction of asthma-like inflammation and stimulating of the immune response. Concentrations of 0 (negative control), 0.5, 1, and 10 and 50 μMolar may be sufficient to determine whether EGS are taken up by cells and functional in the murine lung using confocal microscopy. Variables to be assessed may include presence of absence of toxic effects, cell types with evidence of EGS uptake, dependence on lipid carriers such as Lipofectin and Lipofectase, and functional effects using co-staining of cells with a monoclonal antibody recognizing the murine target of the heterologous sequence and/or in situ cDNA hybridization. The efficacy of the EGS can be monitored by measuring the amount of the target gene mRNA (e.g. using real time PCR) or the amount of polypeptide encoded by the target gene miRNA (Western blot analysis). Cell based assays as described in the Examples below can also be used to analyze the effect of the EGS. Pathology in these animals with the EGS, and animals deficient in the EGS, as well as other suitable controls can be determined through staining of murine lungs and other tissues post-mortem. The efficacy of treatment can be determined by comparing the cellular effects the EGS has in comparison with the EGS deficient animals.

The EGS can be administered directly to humans or animal hosts. For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water. Also, the EGS can be expressed in plants for feeding to both animals and humans.

The compounds of the present disclosure can also be administered to a patient in combination with other therapeutic compounds to increase the overall therapeutic effect. The use of multiple compounds to treat an indication can increase the beneficial effects while reducing the presence of side effects.

### EXAMPLES

### Example #1

### Inhibition of miR-155 by EGS

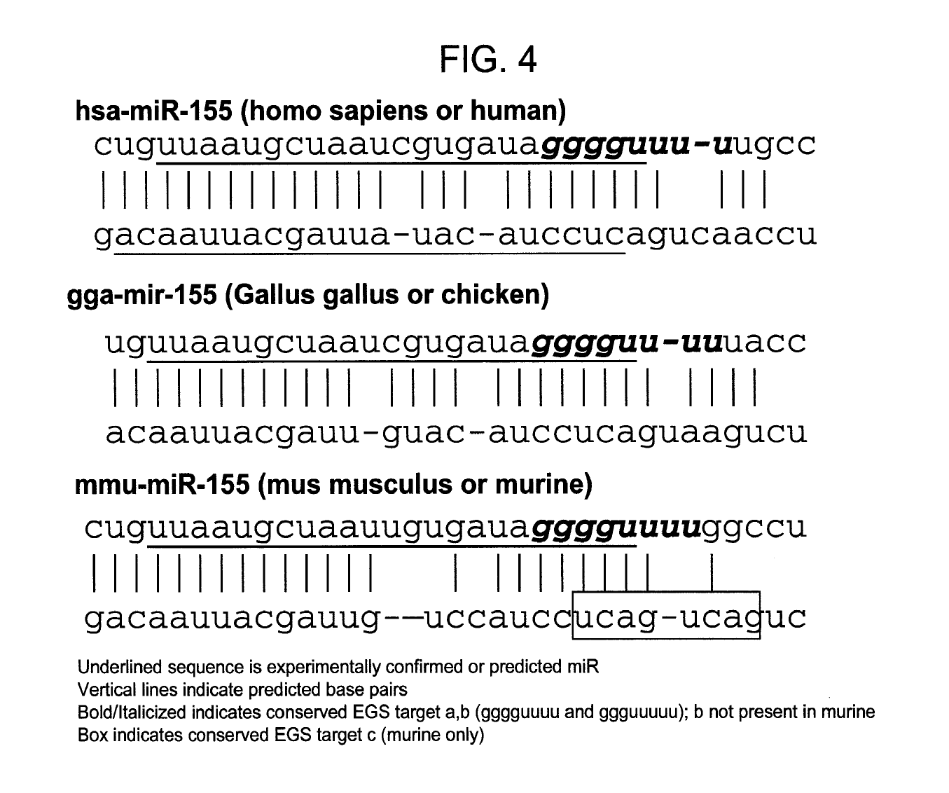

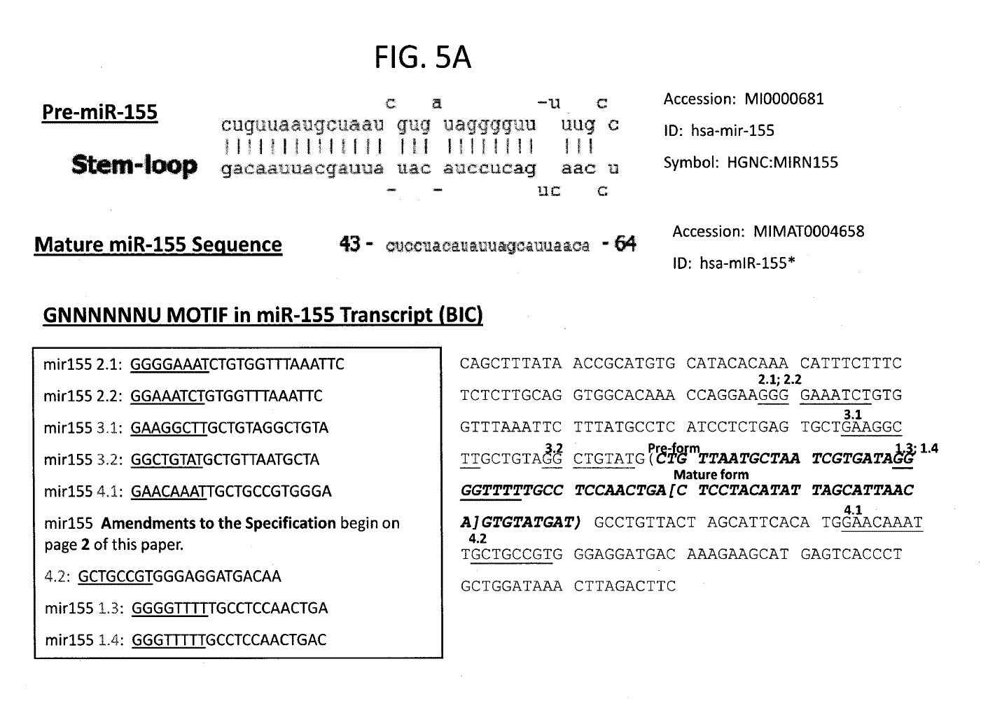

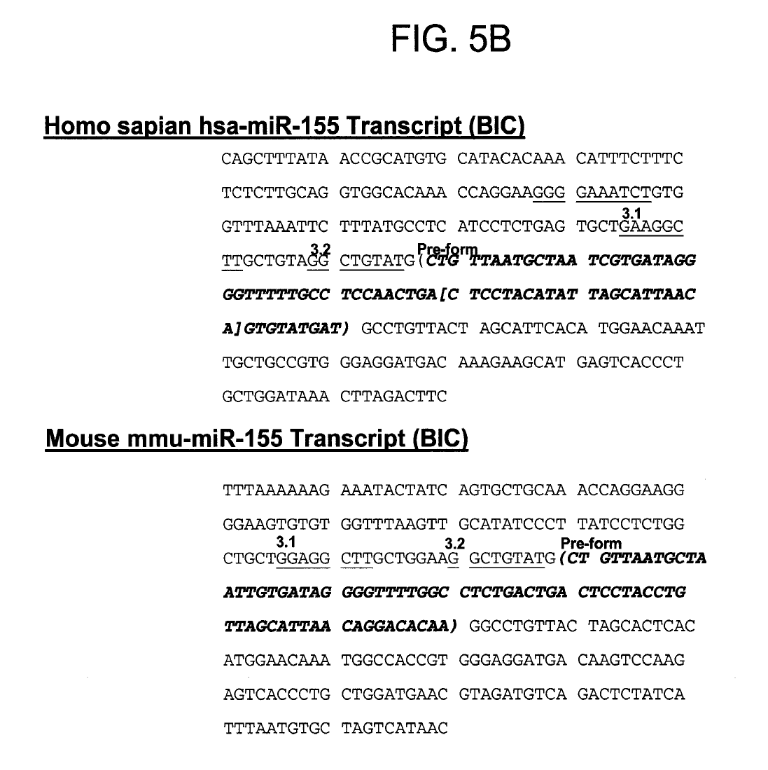

Conserved regions of miR-155 matching RNAse P consensus GNNNNNU within regions of the miRNA precursor/exposed regions of the transcript accessible to RNAse P/EGS were identified (FIG. 4). EGS is designed to be complementary to the GNNNNNU consensus.

The EGS construct sequences are:

WT Long DN miR-155 1.3 WtL (SEQ ID NO. 1):
5′-GGAGGCGTGCGGTCTCCGCGCGCAGGTTCAAATCCTGCAAAC
CCC-3′
Mut Long DN miR-155 1.3 MutL (SEQ ID NO. 2):
5′-GGAGGCGTGCGGTCTCCGCGCGCAGGAAGAAATCCTGCAAAC
CCC-3′
WT Short DN miR-155 1.3 WtS (SEQ ID NO. 3):
5′-GGAGGCGCAGGTTCAAATCCTGCAAACCCC-3′
Mut Short DN miR-155 1.3 MutS (SEQ ID NO. 4):
5′-GGAGGCGCAGGAAGAAATCCTGCAAACCCC-3′
WT Short DN miR-155 3.1 WtS (SEQ ID NO. 5):
5′-CCTACAGGCAGGTTCAAATCCTGCAGCCTTCA-3′
Mut Short DN miR-155 3.1 MutS (SEQ ID NO. 6):
5′-CCTACAGGCAGGAAGAAATCCTGCAGCCTTCA-3′
WT Long DN miR-155 3.1 WtL (SEQ ID NO. 7):
5′-CCTACAGGTGCGGTCTCCGCGCGCAGGTTCAAATCCTGCAGCC
TTCA-3′
Mut Long DN miR-155 3.1 MutL (SEQ ID NO. 8):
5′-CCTACAGGTGCGGTCTCCGCGCGCAGGAAGAAATCCTGCAGCC
TTCA-3′
WT Short DN miR-155 3.2 WtS (SEQ ID NO. 9):
5′-CATTAACAGGCAGGTTCAAATCCTGCTACAGCCA-3′
Mut Short DN miR-155 3.2 MutS (SEQ ID NO. 10):
5′-CATTAACAGGCAGGAAGAAATCCTGCTACAGCCA-3′
WT Long DN miR-155 3.2 WtL (SEQ ID NO. 11):
5′-CATTAACAGGTGCGGTCTCCGCGCGCAGGTTCAAATCCTGCTACA
GCCA-3′
Mut Long DN miR-155 3.2 MutL (SEQ ID NO. 12):
5′-CATTAACAGGTGCGGTCTCCGCGCGCAGGAAGAAATCCTGCTACA
GCCA-3′

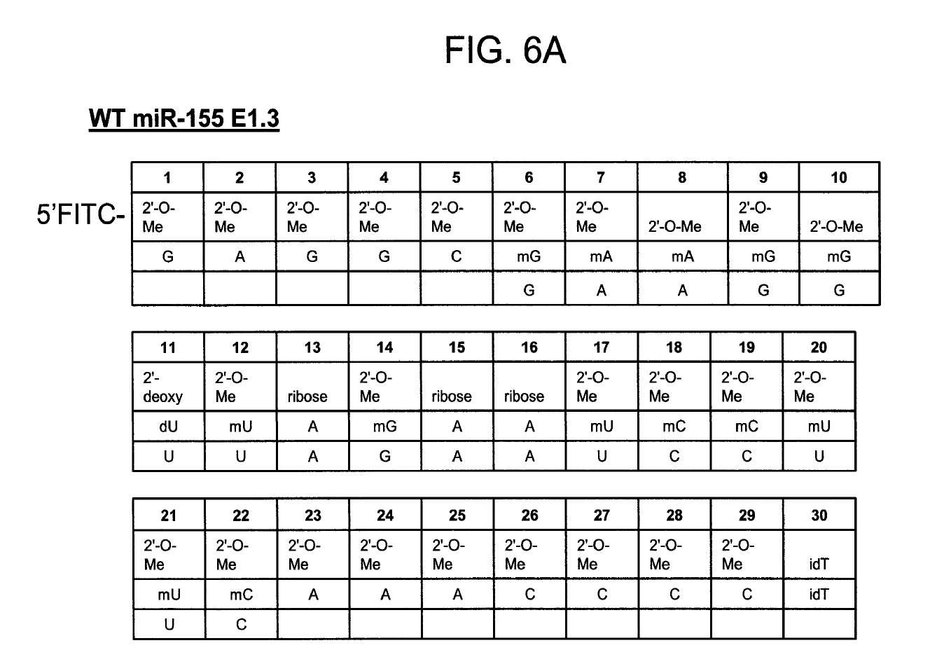

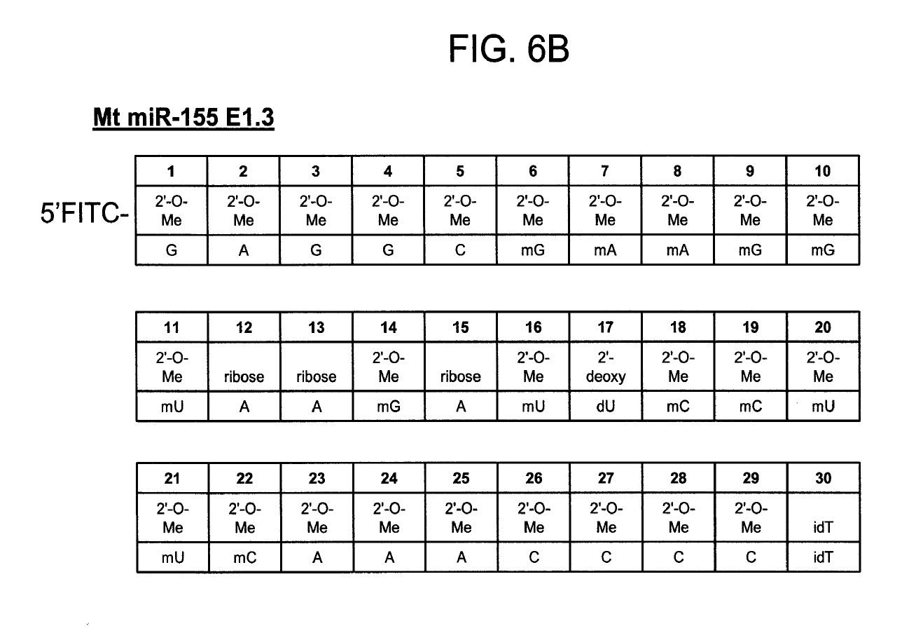

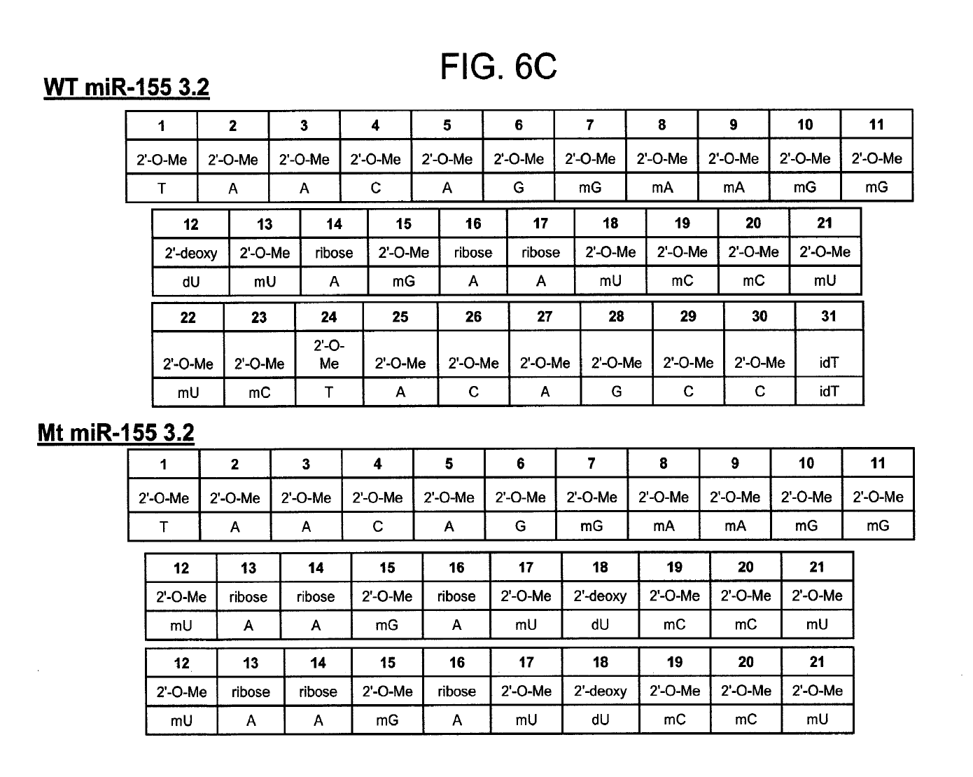

Sequences of RNA-based EGS are depicted in FIG. 6.

A549 cells were plated near confluence in 24-well plates 2 days before transfections were performed using Lipofectamine 2000 (4 microL/well). The amount of DNA (in microliters) is indicated in Table 2 with the concentrations in Table 1 below. All EGSs were added simultaneously with the DNA plasmids in the amounts shown in Table 2. (EGS stock concentrations were all 20 μM in water)

TABLE 1
DNA Constructs

| DNA Constructs | Concentration | Amt. | Source/Reference |
|---|---|---|---|
| BIC | 0.5 ug/ul | See Table 2 | Chung, et al., Nucleic Acids |
| | | | Research, 2006, Vol 34, No. 7 e53 |
| miR-155-Luc | 0.5 ug/ul | See Table 2 | Chung, et al., Nucleic Acids |
| | | | Research, 2006, Vol 34, No. 7 e53 |
| pRL-SV40 | 1.0 ug/ul | 2 ul/well | Renilla luciferase, Promega |

TABLE 2
Amount of Plasmids Used

| | | | miR- | | | |
|---|---|---|---|---|---|---|
| | | | 155- | EGS | EGS | |
| No. | | BIC* | Luc* | wt* | mut* | Description |
| 1 | Control | 0 | 5 | 0 | 0 | Basal miR-Luc activity |
| 2 | wtS DN miR-155 1.3 | 0 | 5 | 10 | 0 | Control for endogenous miR- |
| | | | | | | 155 |
| 3 | mutS DN miR-155 1.3 | 0 | 5 | 0 | 10 | Negative control for No. 2 |
| 4 | Addition of miR-155 encoded | 10 | 5 | 0 | 0 | Inhibit miR-155-Luc activity |
| | in BIC transcript | | | | | |
| 5 | wtS DN miR-155 1.3 | 10 | 5 | 10 | 0 | Reverse inhibition of miR-155 |
| | | | | | | if active |
| 6 | mutS DN miR-155 1.3 | 10 | 5 | 0 | 10 | Control for No. 5 |
| 7 | wtS DN miR-155 3.2 | 10 | 5 | 10 | 0 | Reverse inhibition of miR-155 |
| | | | | | | if active |
| 8 | mutS DN miR-155 3.2 | 10 | 5 | 0 | 10 | Control for No. 6 |

*volume in uL of constructs/plasmids used (concentrations as in Table 1)

Transfections were monitored using a separate set of cells transfected with GFP. After 48 hrs., cells were harvested in 100 ul of 1× Passive Lysis Buffer (Promega). Lysates were analyzed using the Dual Luciferase Assay System (Promega) according to manufacturer's directions.

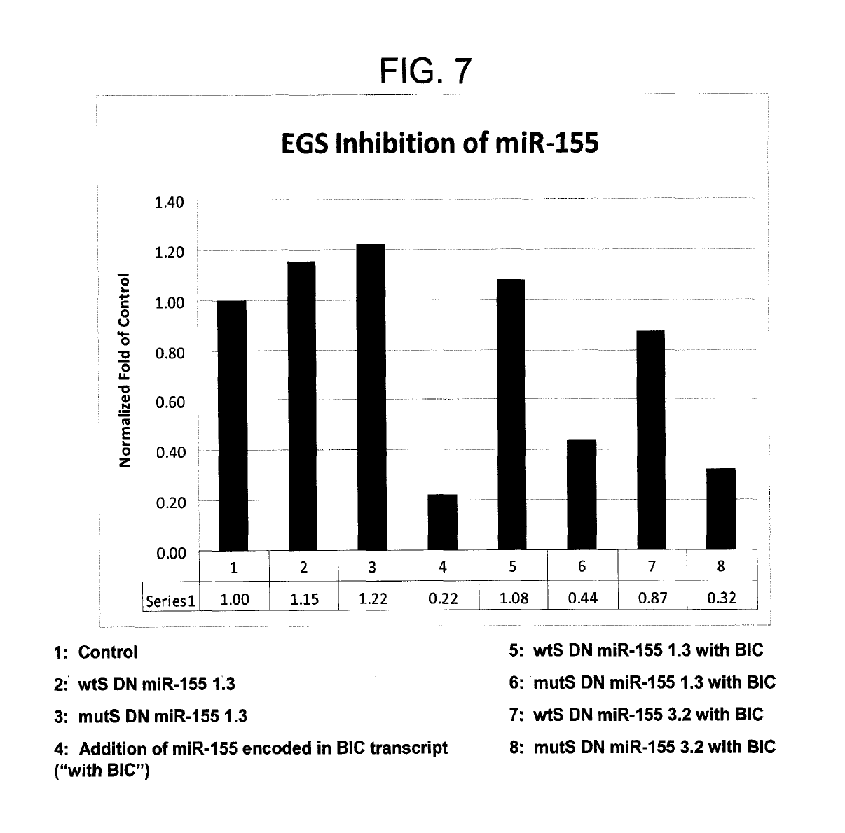

As shown in FIG. 7, the first bar (#1) shows basal luciferase activity expressed from miR-155 Luc, which is a construct containing a binding site for miR-155 contiguous with luciferase. Binding of miR should inhibit the luciferase activity and this is shown in bar #4. Addition of BIC (the transcript of which encodes miR-155) results in loss of luciferase activity (#4). The second and third bars (#2, #3) control for endogenous miR-155 activity. There does not appear to be a significant level of miR-155-basally expressed in A549 cells (as shown by #2, where EGS inhibiting miR-155 is added) since addition of EGS does not increase luciferase activity significantly.

Addition of EGS constructs targeting miR-155 results in restoration of luciferase activity as shown in #5 and #7. The corresponding controls (T-loop mutants of the respective constructs) is shown in #6 and #8. Therefore, miR-155 can be inhibited by these EGS constructs.

### Example #2

### Design of EGS Targeting Mitochondrial mRNA

In this example, EGS is designed to downregulate mitochondrial mRNA. The EGS is designed to form structures resembling precursors to a human tRNA when bound to mitochondrial mRNA based upon standard Watson-Crick base pairing. EGS targeting mitochondrial mRNA is transcribed using T7 RNA polymerase and DNA templates are generated by PCR amplification of a cloned wild type tyrosine tRNA cDNA.

Terminal phosphate 5′ phosphates are added to the oligonucleotides using T4 Polynucleotide Kinase prior to PCR to facilitate blunt end cloning of amplification products. PCR is performed and EGS is subcloned by blunt ended ligation into a plasmid and nucleotide sequence confirmed. The plasmid containing the EGS template, prior to transcription with T7 polymerase, linearized. DNA templates are removed by digestion with RNAse-free DNAse, and RNA transcripts of predicted size should be evident without degradation when viewed on 3% ethidium stained agarose gels prior to incubation with target RNA. The promoter for T7 RNA polymerase is fused to the 5′ region of the EGS cDNA in order to express the EGS in vitro.

To create a regulatable EGS, a U6 promoter, which can directly transcribe small RNAs is used. Existing inducible expression systems are easily modified to include the U6 promoter (Kovrigina et al., RNA 11:1588-1595 (2005)). The pIND (Invitrogen Ecdysone-Inducible Expression system) vector is modified to include the U6 promoter. The EGS sequence described above is cloned into the modified pIND vector with the U6 promoter. The use of the ecdysone analog, ponasterone A, is used to activate transcription of the EGS in mammalian cells.

### Example #3

### In Vitro Cleavage Assay of Mitochondrial mRNA

An in vitro assay for site-specific cleavage of mitochondrial mRNA is prepared by end labeling and purifying a defined 32P labeled fragment of the mitochondrial mRNA transcribed from a plasmid containing mitochondrial cDNA. The labeled mRNA fragment and purified EGS RNA (for example from Example #2) is incubated with the presence of purified RNAse P under conditions described previously (Plehn-Dujowich and Altman, PNAS USA 95:7327-7332 (1998)) and should yield the same fragments as the positive control, RNAse P and control-labeled tRNA, that is incubated under identical conditions. Incubation of EGS RNA and labeled mRNA fragment, or RNAse P and the labeled mRNA fragment, serve as negative controls. Radioactively labeled mRNA substrate or fragments are analyzed by gel electrophoresis (6% polyacrylamide/8M urea sequencing gel). Fragments of mRNA encoding mitochondrial mRNA should increase with increasing ratio of EGS molar ratio to target mRNA when incubated with a constant concentration of RNAse P. Cleavage is expected to be dependent upon concentration of EGS with apparent saturation of the reaction at approximately 1000:1 ratio of EGS to target.

### Example #4

### Cell-Based Assays of a Library of EGS Targeting Genes Involved in the Inflammatory Response

To test the library of EGS targeting an inflammation response in cells, the effect of EGS is compared to cells transfected with empty vector. The effect of the EGS is detected by various means. Northern blotting is used to detect the mRNA levels. Western blotting is used to detect the effect at the protein level. Reporter assays are also used if the mRNA levels and proteins levels are not easily detected by Northern or Western blotting. The downstream effectors of the EGS target are also used to detect the effect of the EGS.

Cells transfected with empty vector show a lower level of target expression at the miRNA, mRNA and/or protein (or its reporter or downstream target) level as compared to cells transfected with the plasmid containing the EGS. Stability and quantity of retained EGS are assessed by sequence analysis of EGS recovered from cells using PCR with primers specific for the 5′ and 3′ termini of EGS. Evidence of integration of EGS into the host genome is determined using PCR of genomic DNA with one primer specific for EGS and a second for host repetitive sequences and Southern blotting of whole chromosomes separated by pulsed field electrophoresis and probed with labeled EGS.

Epithelial and lymphoblastoid cell lines are used in cell based assays. A number of characterized human IL4 and IL13 responsive cell epithelial cell lines are available both from the ATCC (American Type Culture Collection). Human Jurkat, human T-lymphoblastoid and Ramos B-lymphoblastoid cell lines responsive to IL4 and other lymphokines are also available. Human bronchial cells such as BEAS-2B are also be used.

The immune response of the cells is analyzed for molecules involved in inflammation such as those activated through Toll receptors or activation of cellular apoptosis pathways through p53/p21. This analysis uses a combination of gene chips, specific PCR of relevant genes, ELISA, Northern blotting, Western blotting, and/or EMSA to look for altered expression or function of IL-4Rα as well as other inflammatory molecules such as IFN-γ, IL-4, IL-5, and key regulatory proteins and transcription factors such as p21 and NF-κB. Gene chip whole genome screens are readily (more than 20,000 expressed sequence tags and controls, Affymetrix, Santa Clara, Calif.) and are well known to one of ordinary skill in the art. Specific gene chips for 100-150 inflammatory cytokines and receptors (OligoGEArray, Superarray Bioscience, Frederick Mass.) and approximately 250 cellular apoptosis and developmental genes (DualChip, Eppendorf) are also readily available. Custom DNA chips can also be designed and produced by (Affymetrix, Santa Clara, Calif.) and there are highly sensitive culture based assays for inflammatory cytokine production at the protein level (Elispot, Cell Sciences, Canton, Mass.). Other sensitive measures in both non-inflammatory and inflammatory cell states are known to one of ordinary skill in the art.

Cells treated with EGS should have a decreased inflammatory response in comparison to that of cells treated with empty vector. The inflammatory response is determined as mentioned above, by the expression of molecules such as inflammatory cytokines and receptors.

The present disclosure is not limited to the embodiments described above, but is capable of modification within the scope of the appended claims. Those skilled in the art will recognize, or be able to ascertain using no more than routine experimentation, many equivalents of the specific embodiments of the disclosure described herein. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. An external guide sequence (EGS) comprising an oligonucleotide designed to target an immature miRNA.

2. The EGS of claim 1, wherein the immature miRNA is a pri-miRNA or pre-miRNA.

3. An external guide sequence (EGS) comprising an oligonucleotide designed to target a mature miRNA.

4. The EGS of claim 1 or 3, wherein the EGS comprises a subcellular localization sequence.

5. The EGS of claim 4, wherein the subcellular localization element is a nuclear localization element.

6. The EGS of claim 5, wherein the nuclear localization element is a hexamer sequence.

7. The EGS of claim 4, wherein the subcellular localization sequence is a mitochondrial localization sequence.

8. The EGS of claim 7, wherein the mitochondrial localization sequence comprises a peptide or oligonucleotide.

9. The EGS of claim 1, wherein the immature miRNA encodes a miRNA that regulates apoptosis, fat metabolism, development, differentiation, proliferation, or stress response.

10. The EGS of claim 1, wherein the immature miRNA encodes a miRNA that is overexpressed in an immune disease, neurological disease, developmental disease, cardiovascular, skeletal disease or cancer.

11. The EGS of claim 10, wherein the cancer is leukemia, lymphoma, gastric cancer, lung cancer, or prostate cancer.

12. The EGS of claim 1, wherein the immature miRNA encodes a miRNA that is overexpressed and secreted by tumor cells.

13. The EGS of claim 1, wherein the immature miRNA is from a miR-17-92 cluster.

14. The EGS of claim 1, wherein the immature miRNA is from a miR-106-363 cluster.

15. The EGS of claim 1, wherein the immature miRNA encodes miR-21, miR-150, miR-155, or miR-375.

16. The EGS of claim 1, wherein the immature miRNA encodes miR-1-1, miR-1-2, or miR-133.

17. The EGS of claim 1, wherein the immature miRNA encodes a viral miRNA.

18. The EGS of claim 17, where the viral miRNA is from rotavirus, influenza virus, parainfluenza virus, respiratory synctyial virus, herpes virus, Flavivirus, human immunodeficiency virus, hepatitis virus, human papillomavirus, Epstein-Barr virus, Ebola virus, Rous sarcoma virus, human rhinovirus, Variola virus, and poliovirus.

19. The EGS of claim 18, wherein the influenza virus is influenza A, influenza B, or influenza C.

20. The EGS of claim 18, wherein the influenza virus is of the H1N1, H2N2, H3N2, H5N1, H7N7, H1N2, H9N2, H7N2, H7N3, or H10N7 serotype.

21. The EGS of claim 18, wherein the herpes virus is Kaposi's sarcoma herpes virus.

22. A method of modulating gene expression in a host cell comprising:
contacting the host cell with an EGS comprising an oligonucleotide designed to target an immature miRNA, wherein contact with the EGS causes a change in expression of a gene in the host cell in comparison to expression of the gene in a host cell not in contact with the EGS.

23. An external guide sequence comprising an oligonucleotide designed to target a mitochondrial RNA.

24. The EGS of claim 23, wherein the oligonucleotide comprises a mitochondrial localization sequence.

25. The EGS of claim 24, wherein the mitochondrial localization sequence comprises a peptide or an oligonucleotide.

26. The EGS of claim 23, wherein the mitochondrial RNA is derived from a mitochondrial gene causing a dysfunctional electron transport chain.

27. The EGS of claim 23, wherein the mitochondrial RNA is derived from a mitochondrial gene causing a disease of the brain, muscle, nerve, heart, pancreas, eye, ears, kidney, or gastrointestinal system.

28. A method of modulating gene expression in a host cell comprising:
contacting the host cell with an EGS comprising an oligonucleotide designed to target a mitochondrial RNA, wherein contact with the EGS causes a change in expression of a gene in the host cell in comparison to expression of the gene in a host cell not in contact with the EGS.

29. A vector comprising the external guide sequence of claim 1, 3, or 23.

30. The vector of claim 29, wherein the vector comprises a regulatory element.

31. A host cell comprising the external guide sequence of claim 1, 3, or 23.

32. A host cell comprising the vector of claim 29.

33. A library of EGS comprising a plurality of oligonucleotides designed to target a plurality of miRNA.

34. The library of claim 33, wherein the plurality of miRNA comprises immature miRNA.

35. The library of claim 33, wherein the plurality of miRNA comprises mature miRNA.

36. A method of identifying an EGS that modulates expression of a gene comprising:
a) contacting a host cell with a library of EGS comprising a plurality of oligonucleotides designed to target a plurality of miRNA in the host cell;
b) analyzing a gene expression profile of the host cell to determine the gene whose expression is modulated by contact with the library of EGS; and
c) identifying the EGS within the library that modulates the expression of the gene.

37. A library of EGS comprising a plurality of oligonucleotides designed to target a plurality of mitochondrial RNA.

38. A method of identifying an EGS that modulates expression of a gene comprising:
a) contacting a host cell with a library of EGS comprising a plurality of oligonucleotides designed to target a plurality of mitochondrial RNA in the host cell;
b) analyzing a gene expression profile of the host cell to determine the gene whose expression is modulated by contact with the library of EGS; and
c) identifying the EGS within the library that modulates the expression of the gene.

39. The method of claim 36 or 38, further comprising providing analysis or identification of the EGS to an individual.

40. The method of claim 39, wherein providing analysis or identification of the EGS to an individual comprises transmission of the data relating to the analysis or identification over a network.
